(19) United States
(12) Patent Application Publication
(10) Pub. No.: US 2025/0339122 A1
(43) Pub. Date: Nov. 6, 2025

(54) TECHNIQUE FOR TRACKING FLOW USING ULTRASOUND

(71) Applicant: Becton, Dickinson and Company, Franklin Lakes, NJ (US)

(72) Inventors: Julio Agustin Isla Garcia, Irvine, CA (US); Andres S. Aguirre, Mission Viejo, CA (US); Robert Bruce Alexander Adamson, Halifax (CA)

(21) Appl. No.: 19/265,966

(22) Filed: Jul. 10, 2025

Related U.S. Application Data
(63) Continuation of application No. PCT/US2024/011077, filed on Jan. 10, 2024.
(60) Provisional application No. 63/479,256, filed on Jan. 10, 2023.

(51) Int. Cl. A61B 8/06; A61B 8/00

(52) U.S. Cl. CPC A61B 8/06; A61B 8/4488; A61B 8/461; A61B 8/488; A61B 8/5223; A61B 8/54

(57) ABSTRACT

A method for monitoring renal blood flow of a patient includes positioning an ultrasound transducer probe on an abdomen of the patient. The ultrasound transducer probe includes a two-dimensional array of transducer elements. The two-dimensional array of transducer elements and a beamformer driving the two-dimensional array of transducer elements scan a volume of the abdomen of the patient under the field of view of the two-dimensional array of transducer elements. A processor in communication with the ultrasound transducer probe and the beamformer identifies a sub-volume in the volume that includes a Doppler flow signal having a signature of interest corresponding to the renal blood flow of the patient. A set of sequential beams are periodically fired from the two-dimensional array of transducer elements over the sub-volume to track the sub-volume.

### CROSS-REFERENCE TO RELATED APPLICATION(S)

This application is a continuation of International Application No. PCT/US2024/011077, filed Jan. 10, 2024, entitled “TECHNIQUE FOR TRACKING FLOW USING ULTRASOUND,” which claims the benefit of U.S. Provisional Application No. 63/479,256, filed Jan. 10, 2023, and entitled “TECHNIQUE FOR TRACKING FLOW USING ULTRASOUND,” the disclosures of which are hereby incorporated by reference in their entireties.

### BACKGROUND

Acute kidney injury (AKI) occurs when a kidney experiences a sudden decrease in function. AKI can be a complication from major abdominal surgery and may increase a risk of chronic kidney disease in a patient if AKI is not detected and treated at an early stage. Decreased perfusion to the kidney(s) during surgery is one cause of AKI. Detecting AKI in a patient is traditionally done by viewing two biomarkers in the patient. The first biomarker is analyzing urine output of the patient and the second biomarker is measuring serum creatinine from a blood sample of the patient. These biomarkers generally do not show up in the patient until about eight hours to forty-eight hours after the injury has occurred to the kidney(s). Due to the late onset of these biomarkers, physicians can only use these biomarkers to detect whether AKI has occurred a relatively long time after the kidney has been damaged, and cannot use these biomarkers to monitor health of the kidneys in real time during a surgery. The ability to monitor the health of the kidneys and other organs during surgery would not only allow physicians the ability of early detection of AKI, but possibly the ability to prevent AKI in the patient.

### SUMMARY

A method for monitoring renal blood flow of a patient includes affixing an ultrasound transducer probe on an abdomen of the patient, with the ultrasound transducer probe including a two-dimensional array of transducer elements. The two-dimensional array of transducer elements and a beamformer driving the two-dimensional array of transducer elements scan a volume of the abdomen of the patient under the field of view of the two-dimensional array of transducer elements. A processor in communication with the ultrasound transducer probe and the beamformer identifies a sub-volume in the volume that includes a Doppler flow signal having a signature of interest corresponding to the renal blood flow of the patient. A set of sequential beams are periodically fired from the two-dimensional array of transducer elements over the sub-volume to track the sub-volume.

A method for monitoring an organ blood flow of a patient includes affixing an ultrasound transducer probe on an abdomen of the patient. The ultrasound transducer probe includes a two-dimensional array of transducer elements. A finding phase is performed first by scanning, by the two-dimensional array of transducer elements and a beamformer driving the two-dimensional array of transducer elements, a volume of the abdomen of the patient under the field of view of the two-dimensional array of transducer elements. Second, a processor in communication with the ultrasound transducer probe and the beamformer identifies a sub-volume in the volume with a Doppler flow signal having a signature of interest corresponding to the organ blood flow of the patient. A tracking phase of the Doppler flow signal comprising the signature of interest of the organ blood flow is performed by periodically firing a set of sequential beams from the two-dimensional array of transducer elements over the sub-volume.

### BRIEF DESCRIPTION OF THE DRAWINGS

FIG. 1 is a schematic diagram illustrating an example blood flow monitor with an ultrasound transducer probe attached to an abdomen of a patient by an adhesive patch.

FIG. 2 is another schematic diagram illustrating the blood flow monitor of FIG. 1.

FIG. 3 is a schematic diagram of an ultrasound transducer probe attached to an abdomen of a patient by an adhesive patch to monitor a kidney of the patient.

FIG. 4A is another schematic diagram of an ultrasound transducer probe attached to an abdomen of a patient by an adhesive patch to monitor a kidney of the patient.

FIG. 4B is another schematic diagram of an ultrasound transducer probe attached to an abdomen of a patient by an adhesive patch to monitor a kidney of the patient.

FIG. 5 is a schematic diagram of an ultrasound transducer probe with an array of transducer elements.

FIG. 6 includes a standard Power Doppler Imaging (PDI) map at top left, a spatially-filtered version of the standard PDI map at top center, a sub-volume of the spatially-filtered version of the standard PDI map at top right, and a plot of intensity change of a Doppler flow signal from the spatially filtered version of the standard PDI map.

FIG. 7 includes an image of an accumulated PDI map over several frames of the organ blood flow from FIG. 6, a plot at bottom of a depth position of the highest power Doppler signal measured from the accumulated PDI map, and a plot at right of an angle position of the highest power Doppler signal measured from the accumulated PDI map.

FIG. 8A is a schematic diagram of an ultrasound transducer probe scanning a volume of an abdomen of a patient and completing a search of all three dimensions of the volume in a single step.

FIG. 8B is a schematic diagram of an ultrasound transducer probe scanning a volume of an abdomen of a patient and completing a search of a first dimension and a second dimension of the volume in a first step.

FIG. 8C is a schematic diagram of the ultrasound transducer probe of FIG. 8B scanning the volume of the abdomen of the patient and completing a search of a third dimension of the volume in a second step.

FIG. 9 is a schematic diagram of a relative location of a set of precalculated beams tracking an organ blood flow as the organ blood flow shifts positions within a patient.

FIG. 10 is a schematic diagram of relative locations of precalculated beams of the ultrasound transducer probe.

FIG. 11 is a schematic diagram of a relative location of a beam set with a central beam from the precalculated beams of FIG. 10.

FIG. 12 is a schematic diagram of relative locations of precalculated beams of the ultrasound transducer probe.

FIG. 13 is a schematic diagram of a relative location of a beam set with a central beam from the precalculated beams of FIG. 12.

FIG. 14 is a schematic diagram of relative locations of another precalculated beam set of the ultrasound transducer probe that includes precalculated synthetic beams.

### DETAILED DESCRIPTION

The present disclosure is directed to a system and a method to monitor in real time a blood flow of an abdominal organ, such as a kidney, of a patient during a surgery, medical procedure, or medical observation of the patient. The system includes a blood flow monitor with an ultrasound transducer probe. The system also includes an adhesive patch that can attach the ultrasound transducer probe to a patient and keep the ultrasound transducer probe attached to the patient throughout a surgery, medical procedure, or medical observation of the patient without assistance from an ultrasound operator. The blood flow monitor also includes a beamformer and ultrasound front-end (UFE) circuitry in communication with the ultrasound transducer probe to drive an array of transducer elements of the ultrasound transducer probe.

In this disclosure, a Doppler flow signal is defined as comprising an ultrasound pulse-echo signal received from tissue, filtered to only contain those spectral components with a large enough Doppler shift to be reliably identified as having been generated by flowing blood cells. An instantaneous spectrum is defined as a power spectrum of a windowed portion of the Doppler flow signal with a window centered at a particular moment in time. In this disclosure, a Doppler spectrogram is defined as a time-frequency representation of the Doppler flow signal in which instantaneous spectrum is calculated for many timepoints to characterize how the instantaneous spectrum changes over time. The Doppler spectrogram is often visualized as a heat-map plot with frequency along one axis and time along a second axis. Relative intensity of the Doppler spectrogram can be interpreted as an indication of a fraction of scatterers with a particular velocity (i.e. a particular Doppler shift) at a particular moment in time. Negative frequency components of the Doppler spectrogram arise from scatterers that move away from the ultrasound transducer probe while the positive frequency components arise from scatterers moving towards the ultrasound transducer probe. Integrated power spectrum is defined as comprising the integral of the Doppler spectrogram along the frequency dimension. The integral of the Doppler spectrogram may be taken over all frequencies, over only the positive frequencies, over only the negative frequencies or over some other subset of frequencies. In cases where a signal from a particular vessel is sought, the integrated power spectrum will be calculated over a range of frequencies appropriate to isolate the Doppler flow signal from that vessel from interfering signals of nearby vessels. In particular, since blood flow in the renal artery is directed towards the ultrasound transducer probe and blood flow in the renal vein is directed away from the ultrasound transducer probe, the integrated power spectrum calculated in relation to the renal artery can comprise an integral over only positive frequencies while the integrated power spectrum calculated in relation to the renal vein can be calculated only over negative frequencies.

Depending on the application, the system may be configured to measure flow in many multiple different arteries or veins in various organs using the same techniques described in this disclosure for scanning, tracking and measuring Doppler signals. When methods are not specific to a particular vessel, the vessel that is being tracked will be referred to as the target vessel or the target organ blood flow.

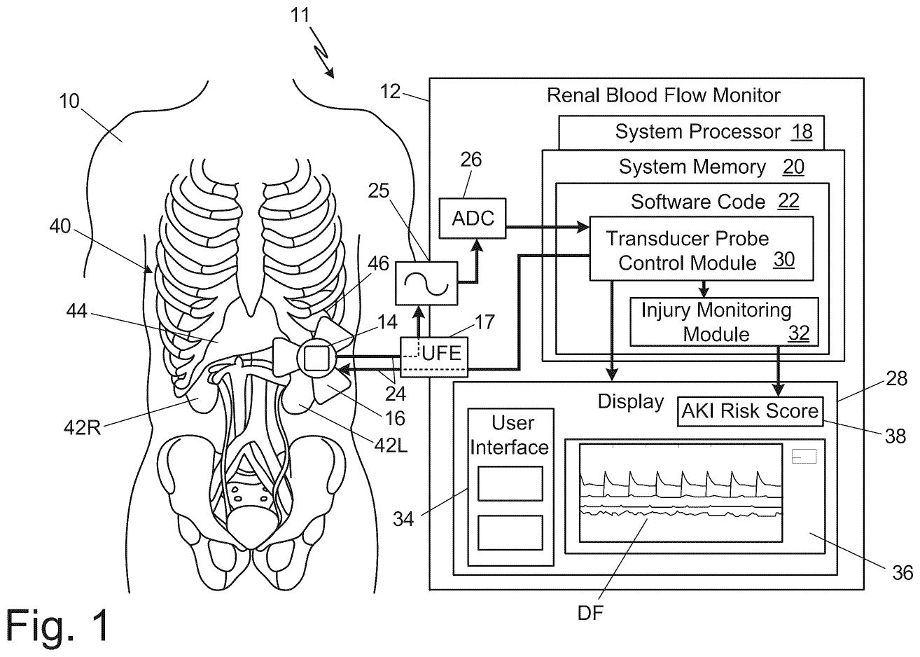

The beamformer is configured to continuously track a Doppler flow signal of an organ blood flow, such as renal blood flow, of the patient by emitting a set of sequential beams from the array of transducer elements and steering them to track the Doppler flow signal of the organ blood flow relative to the array of transducer elements focused on different locations. By beam steering to track the Doppler flow signal of the organ blood flow, the beamformer allows continuous sensing of the Doppler flow signal of the organ blood flow throughout the surgery, medical procedure, or medical observation without moving or readjusting the position of the ultrasound transducer probe on the patient. Even if the organ shifts position in the abdomen of the patient, beam steering by the beamformer enables the ultrasound transducer probe to continue sensing the organ blood flow without moving or readjusting the position of the ultrasound transducer probe on the patient. The ultrasound transducer probe sends the sensed measurements of the Doppler flow signal to the beamformer and the UFE circuitry where the sensed measurements are converted into a real time continuous reading of the organ blood flow. The beamformer and the UFE circuitry send the real time continuous reading of the organ blood flow to the blood flow monitor for health monitoring and perfusion of the organ throughout the duration of the surgery, medical procedure, or medical observation. The blood flow monitoring system is described in detail below with reference to FIGS. 1-14.

FIG. 1 is a schematic diagram of patient 10 and monitoring system 11 that continuously monitors an organ blood flow of patient 10 during a surgery, medical procedure, or medical observation. As shown in the example of FIG. 1, monitoring system 11 can include renal blood flow monitor 12, ultrasound transducer probe 14, adhesive patch 16, ultrasound front-end circuitry 17, system processor 18, system memory 20 with software code 22, probe cables 24, analog-to-digital (ADC) converter 26, and display 28. Software code 22 can include transducer probe control module 30 and injury monitoring module 32. Display 28 can include user interface 34, plot 36, and injury score indicator 38. FIG. 1 also shows abdomen 40 of patient 10 along with kidneys 42L and 42R, liver 44, and spleen 46. In the example of FIG. 1, monitoring system 11 is monitoring a renal blood flow of kidney 42L of patient 10. In other examples, monitoring system 11 can be used to monitor hepatic blood flow of liver 44, to monitor celiac blood flow of spleen 46, the pancreas (not shown) and the stomach (not shown) of patient 10, to monitor mesenteric blood flow of the intestines and/or to monitor portal blood flow from the stomach of patient 10. Thus, renal blood flow monitor 12 can be adapted as an organ blood flow monitor 12 for any organ of patient 10.

Renal blood flow monitor 12, can be, e.g., an integrated hardware unit that includes system processor 18, system memory 20, display 28, ultrasound front-end circuitry 17, and ADC 26. In other examples, any one or more components and/or described functionality of organ blood flow monitor can be distributed among multiple hardware units. For instance, in some examples, display 28 can be a separate display device that is remote from and operatively coupled with renal blood flow monitor 12. In general, though illustrated and described in the example of FIG. 1 as an integrated hardware unit, it should be understood that renal blood flow monitor 12 can include any combination of devices and components that are electrically, communicatively, or otherwise operatively connected to perform functionality attributed herein to renal blood flow monitor 12.

Ultrasound transducer probe 14 can be attached or secured to patient 10 by adhesive patch 16. In the example of FIG. 1, ultrasound transducer probe 14 is positioned on abdomen 40 of patient 10 over at least a portion of kidney 42L. Adhesive patch 16 can include a sheet of structural material, such as fabric or flexible plastic, with a layer of bonding adhesive deposited on a face of the sheet. Adhesive patch 16 can be bonded to or mechanically connected to ultrasound transducer probe 14, or to a frame (not shown) connected to a base of ultrasound transducer probe 14, and can extend outward from ultrasound transducer probe 14 along a surface of abdomen 40 of patient 10. In other examples, adhesive patch 16 can be placed over ultrasound transducer probe 14 to attach ultrasound transducer probe 14 to abdomen 40 of patient 10. Adhesive patch 16 keeps ultrasound transducer probe 14 attached to patient 10 and secured in place throughout a duration of the surgery, medical procedure, or medical observation of patient 10. Since adhesive patch 16 keeps ultrasound transducer probe 14 immobile and in contact with patient 10, an ultrasound operator or technician is not needed during the surgery, medical procedure, or medical observation to keep ultrasound transducer probe 14 in position. A coupling layer (not shown) with a couplant material can be positioned between a skin of patient 10 and ultrasound transducer probe 14. The coupling layer enables ultrasonic energy transmission between the skin of patient 10 and ultrasound transducer probe 14.

In the example of FIG. 1, the ultrasound transducer probe 14 detects and senses a Doppler flow signal DF of the renal blood flow of kidney 42L. Ultrasound transducer probe 14 can be operatively connected to renal blood flow monitor 12 by cables 24. Via cables 24, ultrasound transducer probe 14 can receive electrical signals from the ultrasound front-end circuitry 17 of the renal blood flow monitor 12 and can relay the received ultrasound signals from patient 10 to renal blood flow monitor 12 for extraction of the Doppler flow signal DF of the renal blood flow of kidney 42L. In other examples, ultrasound front-end circuitry 17 is combined with ultrasound transducer probe 14, can be battery powered and can include a receiver to wirelessly receive commands from renal blood flow monitor 12. The combined ultrasound front-end circuitry 17 and ultrasound transducer probe 14 can also include a transmitter to wirelessly communicate the Doppler flow signal DF of the renal blood flow of kidney 42L to renal blood flow monitor 12 for analysis. In some examples, the combined ultrasound transducer probe 14 and ultrasound front-end circuitry 17 provide the Doppler flow signal DF to renal blood flow monitor 12 as analog signal 25, which is converted by ADC 26 to digital hemodynamic data representative of the renal blood flow of kidney 42L. In other examples, the combined ultrasound transducer probe 14 and ultrasound front-end circuitry 17 can provide the sensed Doppler flow signal DF to renal blood flow monitor 12 in digital form, in which case renal blood flow monitor 12 may not include or utilize ADC 26. In yet other examples, ultrasound transducer probe 14 can provide the Doppler flow signal DF of the renal blood flow of kidney 42L to blood flow monitor 12 as analog signal 25, which is analyzed in its analog form by blood flow monitor 12.

System memory 20 can be configured to store information within renal blood flow monitor 12 during operation. System memory 20, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 20 can include volatile and non-volatile computer-readable memories. Examples of volatile memories can include random access memories (RAM), dynamic random access memories (DRAM), static random access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

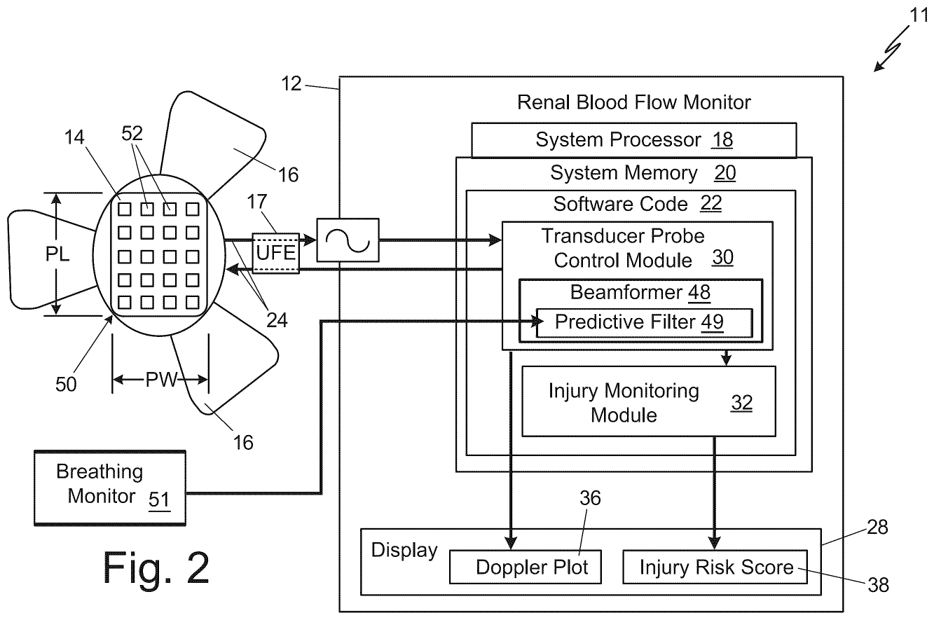

As shown in FIG. 1, system memory 20 of renal blood flow monitor 12 can store software code 22 which forms a monitoring model of renal blood flow monitor 12. Software code 22 can include transducer probe control module 30 for controlling and commanding ultrasound transducer probe 14. Transducer probe control module 30, as discussed in greater detail below with reference to FIG. 2, includes a beamformer that keeps ultrasound transducer probe 14 aimed at the renal blood flow of kidney 42L so that ultrasound transducer probe 14 continuously senses and communicates the Doppler flow signal DF of the renal blood flow to renal blood flow monitor 12 throughout the surgery, medical procedure, or medical observation of patient 10. Software code 22 can also include injury monitoring module 32 which includes acute kidney injury (AKI) monitoring software code and/or specific organ injury (SOI) monitoring software code. This code is monitoring software code that allows injury monitoring module 32 to determine, in real time, a characteristic of the renal blood flow of patient 10, monitor the characteristic of the renal blood flow over time, and determine an AKI risk score of patient 10 from the characteristic and the Doppler flow signal DF of the renal blood flow of kidney 42L. The AKI risk score represents the probability that kidney 42L is experiencing or approaching an AKI. When monitoring system 11 is used to monitor an organ other than kidneys 42L and 42R of patient 10, injury monitoring module 32 can be adapted to determine a real-time organ injury risk score from the Doppler flow signal of the organ blood flow of the organ that is being monitored, such as liver 44.

System processor 18 is a hardware processor configured to execute software code 22, which implements transducer probe control module 30 and injury monitoring module 32, to continuously sense the Doppler flow signal DF and monitor the Doppler flow signal for AKI of kidney 42L. Examples of system processor 18 can include any one or more of a microprocessor, a controller, a digital signal processor (DSP), an application specific integrated circuit (ASIC), a field-programmable gate array (FPGA), or other equivalent discrete or integrated logic circuitry.

Display 28 provides user interface 34, which includes control elements that enable user interaction with renal blood flow monitor 12 and/or other components of monitoring system 11. Display 28 is in communication with system processor 18 and is configured to provide plot 36 in real time of the Doppler flow signal DF of the renal blood flow of kidney 42L. In addition to showing plot 36 of Doppler flow signal DF, display 28 can also provide an audible representation of Doppler flow signal DF via a speaker. Display 28, as shown in FIG. 1, also shows an injury score indicator 38, which is a representation of the real-time AKI risk score of patient 10 determined from the Doppler flow signal DF by system processor 18 and injury monitoring module 32. Display 28 can also include a sensory alarm to alert medical personnel when the real-time AKI risk score of patient 10 is approaching or exceeding a predetermined threshold. The sensory alarm can be implemented as one or more of a visual alarm, an audible alarm, a haptic alarm, or other type of sensory alarm. For instance, the sensory alarm can be invoked as any combination of flashing and/or colored graphics shown by user interface 34 on display 28, a warning sound such as a siren or repeated tone, and a haptic alarm configured to cause renal blood flow monitor 12 to vibrate or otherwise deliver a physical impulse perceptible to medical personnel.

Display 28 can be a liquid crystal display (LCD), a light-emitting diode (LED) display, an organic light-emitting diode (OLED) display, or other display device suitable for providing information to users in graphical form. User interface 34 can include graphical and/or physical control elements that enable user input to interact with renal blood flow monitor 12 and/or other components of monitoring system 11. In some examples, user interface 34 can take the form of a graphical user interface (GUI) that presents graphical control elements presented at, e.g., a touch-sensitive and/or pressure sensitive display screen of display 28. In such examples, user input can be received in the form of gesture input, such as touch gestures, scroll gestures, zoom gestures, or other gesture input. In certain examples, user interface 34 can take the form of and/or include physical control elements, such as a physical buttons, keys, knobs, or other physical control elements configured to receive user input to interact with components of monitoring system 11. User interface 34 can include a speaker that allows renal blood flow monitor 12 the ability to generate an audible alarm.

In operation of monitoring system 11, before a surgery, medical procedure, or medical observation begins, a medical worker places ultrasound transducer probe 14 on abdomen 40 of patient 10. The medical worker uses ultrasound transducer probe 14 to locate the Doppler flow signal DF of the renal blood flow of kidney 42L. Ultrasound transducer probe 14 can generate an audible representation of the Doppler flow signal DF to assist the medical worker in locating the Doppler flow signal DF of the renal blood flow of kidney 42L. Once the medical worker finds the Doppler flow signal DF of the renal blood flow of kidney 42L, the medical worker attaches and secures ultrasound transducer probe 14 to patient 10 with adhesive patch 16. Adhesive patch 16 keeps ultrasound transducer probe 14 in constant contact with patient 10 such that ultrasound transducer probe 14 does not shift positions on patient 10 during the surgery, medical procedure, or medical observation and lose the Doppler flow signal DF of the renal blood flow of kidney 42L. Ultrasound transducer probe 14 relays the received ultrasound signals to renal blood flow monitor 12 via cable(s) 24 or wirelessly. In the case of wireless transmission, the ultrasound transducer probe 14 includes the ultrasound front-end circuitry 17. System processor 18 of renal blood flow monitor 12 receives the Doppler flow signal DF and processes the Doppler flow signal DF sequentially or simultaneously through transducer probe control module 30 and injury monitoring module 32.

System processor 18 can execute the AKI monitoring software code of injury monitoring module 32 to establish a baseline value for the renal blood flow of kidney 42L of patient 10 from the Doppler flow signal DF sensed by ultrasound transducer probe 14. Deviations from the baseline value for the renal blood flow can be used as factors by system processor 18 and injury monitoring module 32 to calculate the real-time AKI risk score of kidney 42L. System processor 18 can further execute the AKI monitoring software code of injury monitoring module 32 to continuously monitor the Doppler flow signal DF of the renal blood flow sensed by ultrasound transducer probe 14 throughout a duration of the surgery, medical procedure, or medical observation of patient 10 and estimates the AKI risk score of kidney 42L of patient 10 from the Doppler flow signal DF. System processor 18 outputs the Doppler flow signal DF and the real-time AKI risk score of kidney 42L to display 28. Display 28 produces plot 36 showing the Doppler flow signal DF of the renal blood flow of kidney 42L plotted over time. Display 28 also produces injury score indicator 38 which represents the real-time AKI risk score of kidney 42L in injury score indicator 38.

As the surgery, medical procedure, or medical observation of patient 10 progresses, system processor 18 continues to receive the Doppler flow signal DF from ultrasound transducer probe 14 and continues to output both the Doppler flow signal DF and the real-time AKI risk score of kidney 42L to display 28. If the real-time AKI risk score of kidney 42L changes toward an undesired threshold, or changes at an undesired rate, system processor 18 and display 28 can alert the medical personnel so that the medical personnel can possibly take action to increase kidney perfusion and prevent AKI to kidney 42L, or minimize AKI to kidney 42L. For example, medical personnel can administer medication or fluids that increases the renal blood flow and perfusion to kidney 42L or improves autoregulation of the renal blood flow to kidney 42L. At the end of the surgery, medical procedure, or medical observation, system processor 18 and injury monitoring module 32 can estimate a final AKI risk score for kidney 42L and output the final AKI risk score to display 28. If the final AKI risk score for kidney 42L indicates that kidney 42L has a high risk of AKI, medical personnel can take immediate action to treat kidney 42L without having to wait for biomarkers to appear in blood and urine samples of patient 10. Biomarkers that indicate AKI can take several hours or days to appear in blood and urine samples of patient 10. With monitoring system 11, the medical personnel can determine quickly whether patient 10 needs to be treated for AKI of kidney 42L.

When the location of kidney 42L changes relative to ultrasound transducer probe 14 due to respiration or movement of patient 10 during the surgery, medical procedure, or medical observation, transducer probe control module 30 will detect a change in the Doppler flow signal DF and will respond by adjusting the focusing location of the set of beams to scan abdomen 40 of patient 10 to relocate the Doppler flow signal DF and aim ultrasound transducer probe 14 at the new location of the Doppler flow signal DF of the renal blood flow of kidney 42L. As discussed below with reference to FIGS. 2-5, renal blood flow monitor 12 can include a beamformer that can steer beam signals produced by an array of transducer elements of ultrasound transducer probe 14.

FIG. 2 is another schematic diagram of renal blood flow monitor 12. As shown in FIG. 2, renal blood flow monitor 12 can include beamformer 48 with predictive filter 49. Ultrasound transducer probe 14 can include array 50 of transducer elements 52. Each transducer element 52 of array 50 can comprise a piezoelectric material, such as lead zirconate titanate, capable of transmitting ultrasound pulses and detecting ultrasound pulses. Array 50 of transducer elements 52 of ultrasound transducer probe 14 can form a two-dimensional phased array with probe length PL and probe width PW. As a phased array, each transducer element 52 in array 50 can pulse individually relative the other transducer elements 52 in array 50. Monitoring system 11 can also include breathing monitor 51, or can be in communication with breathing monitor 51.

In the example of FIG. 2, beamformer 48 drives array 50 of transducer elements 52 via system processor 18 and ultrasound front-end circuitry 17. Beamformer 48 functions as a transducer probe controller with flow signal tracking software code that controls the timing that each transducer element 52 in array 50 emits an ultrasound pulse. Beamformer 48 can time and pattern when each transducer element 52 emits a pulse such that array 50 can form one or more ultrasonic beams and can sweep or steer the one or more ultrasonic beams without physically moving the position of ultrasound transducer probe 14 on patient 10. Beamformer 48 can be a software sub-module of transformer probe control module 30 that can be executed by system processor 18 to control activation of transducer elements 52 of array 50. Predictive filter 49 can be a software sub-module of beamformer 48 and/or transformer probe control module 30 that can be executed by system processor 18 to predict an expected trajectory of a target vessel based on measured inputs from beamformer 48 and/or from inputs from other external sensors, such as breathing monitor 51. In other examples, beamformer 48 can be a separate hardware component from system processor 18 and system memory 20 with separate memory and software from software code 22 that coordinates with system processor 18 to control activation of transducer elements 52 of array 50. In the example of FIG. 2, beamformer 48 is housed within renal blood flow monitor 12 as part of transducer probe control module 30 of software code 22 that is executed by system processor 18. In other examples, beamformer 48 can be fully or partially housed within a casing of ultrasound transducer probe 14 as a separate hardware and software unit that coordinates with system processor 18. Housing beamformer 48 in the same unit as renal blood flow monitor 12 (whether as part of software code 22 or as an add-on hardware component) can decrease the overall size and thickness of ultrasound transducer probe 14. Ultrasound transducer probe 14 can be relatively thin and flat in profile, with a thickness that is smaller than a width or diameter of ultrasound transducer probe 14. Attaching ultrasound transducer probe 14 to patient 10 by adhesive patch 30 is easier and more secure when ultrasound transducer probe 14 has a thin and flat profile.

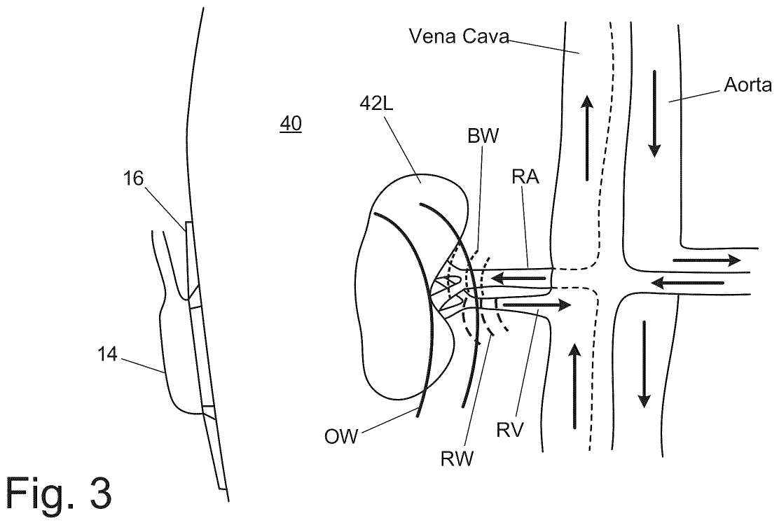

FIG. 3 is another schematic diagram of ultrasound transducer probe 14 attached to abdomen 40 of patient 10 by adhesive patch 16 over kidney 42L. The Doppler flow signal DF of kidney 42L can be measured from either the renal artery RA as blood enters kidney 42L from the aorta of patient 10 via the renal artery or from the renal vein RV as blood exits kidney 42L to the vena cava of patient 10 via the renal vein RV. Ultrasound transducer probe 14 generates originating signals OW that move into abdomen 40 of patient 10. Due to Doppler physics, a Doppler signal BW of the blood flow in the renal artery RA is “blue shifted” as the blood flow in the renal artery RA is moving toward the ultrasound transducer probe 14. A Doppler signal RW of the blood flow in the renal vein RV is “red shifted” as the blood flow in the renal vein RV is moving away from the ultrasound transducer. Since the Doppler signal BW is blue shifted and the Doppler signal RW is red shifted, renal blood flow monitor 12 can easily distinguish renal artery blood flow from renal vein blood flow. In human subjects the renal artery RA and renal vein RV are close and aligned parallel such that beamformer 48 can position the beam(s) to capture both arterial and venous flow of kidney 42L simultaneously.

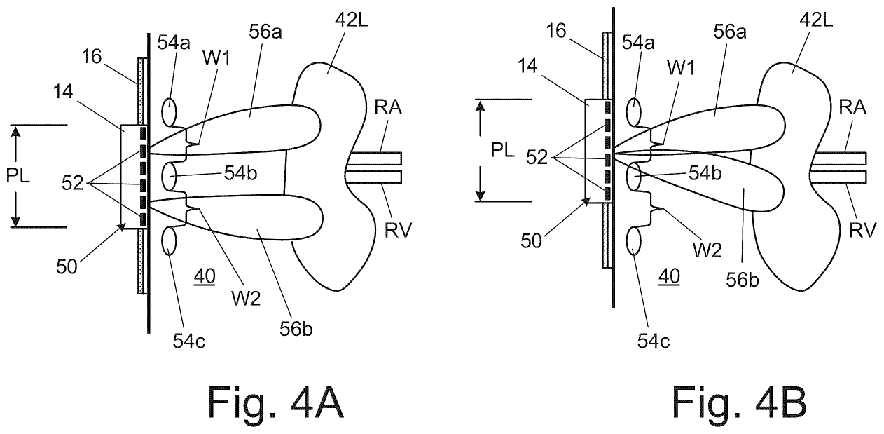

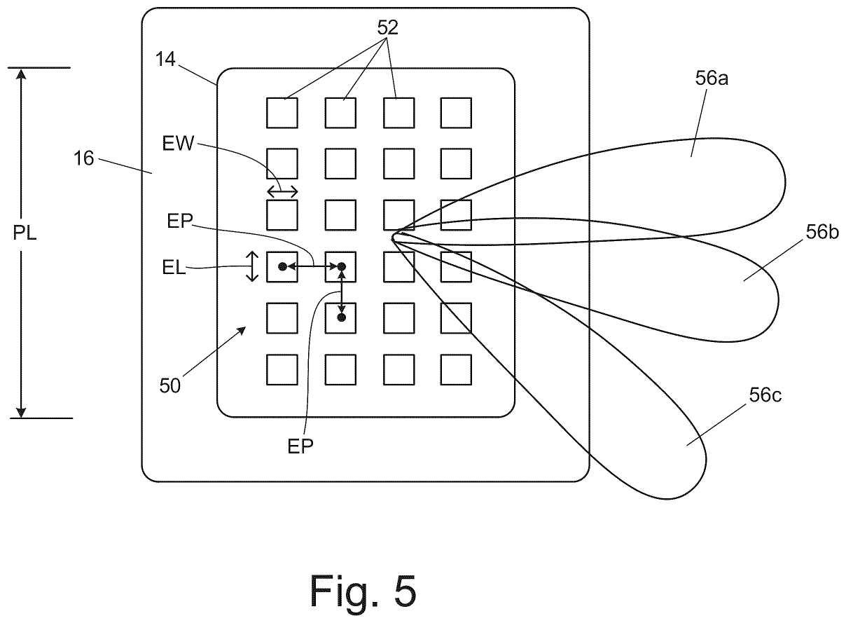

FIGS. 4-5 will be discussed concurrently. FIG. 4A is another schematic diagram of ultrasound transducer probe 14 attached to abdomen 40 of patient 10 by adhesive patch 16 over kidney 42L. FIG. 4B is also a schematic diagram of ultrasound transducer probe 14 attached to abdomen 40 of patient 10 by adhesive patch 16 over kidney 42L. FIG. 5 is another schematic diagram of ultrasound transducer probe 14. In the example of FIGS. 4A and 4B, ultrasound transducer probe 14 is attached by adhesive patch 16 to a surface of abdomen 40 over kidney 42L and over at least some of ribs 54a, 54b, and 54c of patient 10.

Ultrasound transducer probe 14 can include a probe length PL, probe width PW (shown in FIG. 2), or diameter that is large enough that array 50 of transducer elements 52 of ultrasound transducer probe 14 can cover one or more acoustic windows in patient 10. An acoustic window of patient 10 is defined as an area of patient 10 where transmission of ultrasonic waves is not substantially attenuated in comparison to immediate surroundings. For example, array 50 of transducer elements 52 of ultrasound transducer probe 14 can be sized in length or width to extend over at least two intercostal spaces of patient 10. For example, in FIG. 4A, array 50 of transducer elements 52 of ultrasound transducer probe 14 is positioned over first acoustic window W1 (formed by the intercostal space between rib 54a and rib 54b) and over second acoustic window W2 (formed by the intercostal space between rib 54b and rib 54c). In the example of FIG. 4A, beamformer 48 (shown in FIG. 2) can selectively activate transducer elements 52 in array 50 to steer signal beams 56a, 56b, and 56c (not visible) into abdomen 40 through the first acoustic window W1 and/or second acoustic window W2 to avoid ribs 54a, 54b, and 54c. In the example of FIG. 4B, ultrasound transducer probe 14 is positioned slightly higher on abdomen 40 of patient 10 in comparison to the example of FIG. 4A. However, the probe length PL or probe width PW of ultrasound transducer probe 14 is long enough that ultrasound transducer probe 14 still has access to first acoustic window W1 and can still scan and steer signal beams 56a, 56b, and 56c (not visible) into abdomen 40 through the first acoustic window W1. Regardless of where ultrasound transducer probe 14 is placed over ribs 54a, 54b, and 54c, ribs 54a, 54b, and 54c will not block the direct view of kidney 42L from array 50 of ultrasound transducer probe 14.

Beamformer 48 controls transducer elements 52 in array 50 to beam scan abdomen 40 to locate a target vessel when ultrasound transducer probe 14 is first placed on patient 10. To find the target vessel, beamformer 48 divides the entirety of the field of view of array 50 into multiple sub-volumes and uses a predefined set of beams (such as beams 56a, 56b, and 56c) to probe each sub-volume. To reduce the search time, the search can be performed in two steps. In a first step the sub-volumes can be made larger in a depth dimension into abdomen 40 while a two-dimension scan is performed in the other two dimensions only. Once the location of the signal in the other two dimensions is determined by the two-dimension scan, the next step is to reduce the size of the sub-volume in the depth dimension and perform a search along the depth dimension at the previously determined location in the other two dimensions. The sub-volumes can be made larger in the depth dimension by increasing the duration of the driving pulses used to form the firing beams or by extracting the Doppler signal at all depths from the received signal from a single firing beam by adjusting the time delay and selecting the one with the highest intensity or an average of the stronger ones.

Beamformer 48 also controls transducer elements 52 in array 50 to track scan abdomen 40 to track the target vessel over time. Beamformer 48 beam scans and/or track scans the Doppler flow signal DF of the renal blood flow of kidney 42L of patient 10 by sequentially emitting signal beams 56a, 56b, and 56c from array 50 of transducer elements 52 and focusing each of beams 56a, 56b, and 56c in different locations. To track in both the azimuth dimension and the elevation dimension (sometimes referred to as altitude dimension), at least three beams are required. Using more beams will result in more accurate target vessel position estimation at the cost of a lower Nyquist frequency for the Doppler shift and hence the possibility of aliasing of the instantaneous spectrogram. Thus, beamformer 48 is not limited to three beams and can include more than three beams. The beam locations of beams 56a, 56b, and 56c are selected to have a sufficient degree of overlap of beams 56a, 56b, and 56c, such that when a target vessel is located at center of the three beams the signal-to-noise ratio of the Doppler flow signal in each of the beams is acceptably large (e.g. >20 dB). For example, the beam locations may be selected so that the center of beams 56a, 56b, and 56c lies at a point where the pressure is 3 dB below its peak value for each of beams 56a, 56b, and 56c.

By comparing integrated spectral power measured along multiple signal beams (e.g. beams 56a, 56b, and 56c), beamformer 48 and/or renal blood flow monitor 12 can estimate a bearing (i.e. the azimuthal and elevation angles) of the target vessel relative to array 50 of transducer elements 52. As a target vessel (e.g., the renal artery RA, and/or the renal vein RV) moves within abdomen 40, or as ultrasound transducer probe 14 moves relative to the target vessel due to respiration of patient 10, the target vessel will move closer to the focus of some of signal beams 56a, 56b, and 56c, which increases the integrated spectral power measured along those beams, and will move further away from the focus of some other(s) of signal beams 56a, 56b, and 56c, which decreases the integrated spectral power measured along those beams. As the target vessel moves, beamformer 48 can redirect signal beams 56a, 56b, and 56c (and possibly more signal beams) in the direction of those beams for which the measured integrated power spectrum is higher and away from those beams for which the integrated power spectrum is lower, thereby tracking the target vessel whose scatterers generate the Doppler flow signal DF. In one embodiment incorporating this tracking methodology, beamformer 48 computes an estimated location for the target vessel as a vector sum of unit vectors along the signal beam directions weighted by the integrated spectral power measured along each of signal beams 56a, 56b, and 56c. The weighting by the integrated spectral power ensures that as beamformer 48 redirects signal beams 56a, 56b, and 56c to the estimated target vessel location, the centroid of the beams 56a, 56b, and 56c will move towards those beams that have the largest integrated spectral power and therefore lie closest to the target vessel.

In another embodiment, beamformer 48 and/or renal blood flow monitor 12 can include a physical model that predicts the integrated spectral power for a given displacement between a signal beam and a target vessel to improve the estimate of the target vessel location. The model may, for example, calculate the integrated power spectrum as an overlap integral between an assumed beam shape (such as a Gaussian beam, a beam described by a sombrero function, or a beam described by a cardinal sine function, depending on transducer shape and apodization) and an assumed geometry for a target vessel such as a cylindrical vessel with a uniform density of moving scatterers across its cross-section. In some embodiments, the model may incorporate information about the change in beam shape with distance from transducer elements 52 as obtained from empirical measurements or acoustic simulation. In some embodiments the model may use an asymmetric beam shape such as an elliptical Gaussian beam with a narrower dimension and a wider dimension as would be produced by an asymmetric array of transducer elements. To estimate the target vessel location from the integrated power spectrum observed along multiple signal beams, the model is inverted using a standard function inversion methodology such as least-squares fitting, interpolation, series expansion, look-up tables and root- finding methods. Once the inverse function has been approximated, it can be used to obtain an estimate of the vessel target from the integrated spectral power measured along the signal beams.

In some embodiments, beamformer 48 and/or renal blood flow monitor 12 can use estimates of the target vessel location as an input to predictive filter 49, shown in FIG. 2, that contains a model of the expected trajectory of the target vessel. The model of predictive filter 49 can make tracking more stable and accurate by allowing beamformer 48 and/or renal blood flow monitor 12 to infer in advance a new location for the signal beams that maximizes the signal or signals that correspond to the signature of interest of the target vessel. The model of predictive filter 49 can include Kalman filters. For example, in cases where the main source of target vessel motion is from breathing, predictive filter 49 may contain a periodic trajectory model describing the motion as periodic at the breathing frequency. In some embodiments, the periodic trajectory model may be implemented as a partial Fourier sum in each direction with the breathing frequency as the fundamental frequency. In such embodiments, model parameters may include some or all of the amplitude and phase (or equivalently, the amplitudes of the in-phase and quadrature components) of each Fourier component in each direction and the location of the origin about which the periodic motion occurs. In some embodiments, predictive filter 49 allows the model parameters to be updated in response to a target vessel position estimate obtained from the integrated power spectrum along a plurality of signal beams so that drift in the model parameters over time or the failure of the model to fully describe the trajectory may be accommodated.

In some embodiments, predictive filter 49 may incorporate an estimate of uncertainty in the estimate of the target vessel position obtained from the integrated power spectrum measurements. This uncertainty estimate may be used to adjust the degree to which the model parameters are affected by new measurements during parameter updates. In some embodiments, this uncertainty estimate may be used to force monitoring system 11 to ignore measurements that are invalid, due, for example, to a transient event that corrupts measurements over a period of time. In some embodiments, this uncertainty estimate may be used to reduce the degree to which measurements affect model parameters when the signal-to-noise ratio of the integrated power spectrum is low and to increase the degree to which measurements affect model parameters when the integrated power spectrum signal-to-noise ratio is high. In some embodiments, the uncertainty estimate may be adjusted in response to changes in the moments of the instantaneous spectrum of the Doppler flow signal (e.g. the mean velocity, the spectral bandwidth), or the maximum velocity envelope of the Doppler spectrogram. In some embodiments, the uncertainty estimate may be adjusted based on the total integrated power spectrum, including both the negative and positive frequencies, or based on an integrated power spectrum in a different range of Doppler shifts than the range used to estimate target vessel position. For example, the integrated power spectrum over the negative frequencies may be used to estimate the uncertainty in a position estimate arrived at using the integrated power spectrum over the positive frequencies.

The integrated spectral power is an inherently noisy signal as the Doppler spectrogram contains speckle arising from constructive and destructive interference between large numbers of scatterers distributed randomly through the insonified volume of abdomen 40 and from statistical noise due to variance in the number and orientation of scatterers in the beam(s) over time. Additionally, the integrated power spectrum is modulated by the cardiac cycle because a larger fraction of scatterers will have Doppler shifts large enough to pass through the filter that defines the Doppler flow signal during systole than during diastole. If unmitigated, the variability in the integrated power spectrum due to speckle and the cardiac cycle will lead to a noisy estimate of target vessel location and to inaccurate tracking. In some embodiments, the noise on the integrated power spectrum is reduced by applying a filter to the integrated power spectrum signal prior to using the integrated power spectrum signal to estimate the target vessel location. Making a kernel duration of the filter longer will make the filter more effective at removing noise, but if the kernel duration of the filter becomes comparable to a timescale of target vessel motion of the target vessel, then the filter will begin to degrade tracking accuracy. Since the fastest source of the target vessel motion is breathing, a filter kernel size shorter than the breathing cycle duration advantageously reduces modulation from cardiac cycle and speckle when maintaining target vessel location estimation accuracy. Statistical noise and speckle noise produce long-tailed intensity distributions with a high probability of producing very large values. Consequently, because of these occasional very large intensities, linear filters are ineffective at smoothing the integrated power spectrum. Median filters are advantageously insensitive to outliers and provide a smoother output than is possible with linear filters. Consequently, in some embodiments, a median filter is used to filter the integrated power spectrum. In some embodiments the median filter kernel size is selected to be larger than the cardiac cycle duration but less than the breathing cycle duration.

In some embodiments, information obtained from other sensors separate from ultrasound transducer probe 14 or a priori information may also be provided to predictive filter 49 estimating the target vessel location. Predictive filter 49 may be configured to incorporate this additional information when adjusting the model parameters as well as adjusting the estimate of target vessel position obtained from the integrated power spectrum. For example, predictive filter 49 may receive input from breathing monitor 51 connected to patient 10 and may use measurements from breathing monitor 51 to update model parameters that capture a breathing frequency of patient 10. In some embodiments, predictive filter 49 can incorporate both measurements made with external sensors (such as breathing monitor 51) and the estimate of target vessel position obtained from the integrated power spectrum to adjust model parameters. In some embodiments, predictive filter 49 may use information obtained from integrated power spectrum measurements taken at an earlier point in time. For example, in some embodiments, tracking of the target vessel may be halted and the directions of signal beams 56a, 56b, and 56c may be fixed in order to observe the periodicity in the integrated power spectrum as the target vessel moves due to breathing. This observation may be used to estimate breathing frequency so that the breathing frequency may be incorporated into predictive model 49 when tracking resumes.

In some embodiments, predictive filter 49 is implemented as a Linear Kalman Filter. In some embodiments, predictive filter 49 is implemented as an Unscented Kalman Filter. In some embodiments, predictive filter 49 is implemented as an Extended Kalman Filter. An input to the Kalman filter can be a centroid of the estimated location of the target vessel in all three dimensions with the Kalman filter based on a periodic movement model with one or more frequency components. Alternatively, the input can be a centroid of the estimated location of the target vessel in only two dimensions while the depth tracking is achieved by extracting the Doppler signal for all depths from the received signal from a single firing beam using the all-depths approach previously described. In yet another alternative approach, a different Kalman filter can use the integrated power spectrum from all firing beams and be based on a different model that estimates position as a byproduct of predicting the integrated power spectrum.

In some embodiments, predictive filter 49 may be configured to produce an estimate of the integrated power spectrum signal along each of a plurality of signal beams (such as signal beams 56a, 56b, and 56c) based on an internal parametric model of target vessel position, beam shape and target vessel shape and orientation. The estimate of the integrated power spectrum by predictive filter 49 for each of the plurality of signal beams may be compared to measurements of the integrated power spectrum along each signal beam, and the difference between the prediction and measurement can be used to update the model parameters including those describing the target vessel location. In calculating the integrated power spectrum along the plurality of signal beams, predictive filter 49 may make use of a physical model of the integrated power spectrum that calculates an overlap integral between the target vessel and the ultrasound beam profile. In some embodiments the physical model may include a description of how the beam profile changes with depth. In some embodiments the physical model may include an asymmetric beam profile such as would be produced by an asymmetric transducer array.

In some embodiments, differences in integrated power spectrum between the different signal beams 56a, 56b, and 56c are used by beamformer 48 and/or renal blood flow monitor 12 to estimate the bearing (azimuthal and elevation angles) of the target vessel, while the range (distance from the transducer) of the target vessel is estimated by beamformer 48 and/or renal blood flow monitor 12 by calculating the integrated power spectrum at a plurality of range samples, assigning a likelihood of containing the target vessel to each range sample, and calculating an estimate of the center of the target vessel from the plurality of range samples. In some embodiments, the likelihood that a range sample contains the target vessel is made proportional to the integrated power spectrum at that range so that the estimate of the location of the target vessel range may be estimated, for example, by beamformer 48 and/or renal blood flow monitor 12 by selecting the range sample with the largest integrated power spectrum or calculating the location of the centroid over the range samples. In other embodiments, beamformer 48 and/or renal blood flow monitor 12 can use a likelihood function to take into account integrated power spectrum, spectral moments, Doppler spectrogram shape, and/or integrated power in spectral ranges other than the range where the integrated power spectrum is calculated. In many cases, the target vessel may extend over a plurality of range samples, in which case, the accuracy of the integrated power spectrum may be improved by averaging over the plurality of range samples likely to contain the target vessel.

In some embodiments, an estimate of target vessel range incorporates the integrated power spectrum calculated for each of a plurality of signal beams (e.g. 56a, 56b, 56c). In some embodiments, beamformer 48 and/or renal blood flow monitor 12 can arrive at this estimate by first averaging the integrated power spectrum across the plurality of beams at each range sample and then calculating a likelihood of each range sample containing the target on this averaged signal.

Separating the estimate of the bearing of the target vessel from the estimate of the range of the target vessel in this way is advantageous as the beamformer 48 and/or renal blood flow monitor 12 can calculate the range estimations more frequently than the bearing estimations over time. Beamformer 48 and/or renal blood flow monitor 12 can obtain a range estimate on every ultrasound transmit event, while a bearing estimate requires that beamformer 48 move an ultrasound beam to a plurality of locations and that the measurements made at the different locations be compared by beamformer 48 and/or renal blood flow monitor 12. Having a reliable estimate of range associated with each transmit event ensures that when beamformer 48 and/or renal blood flow monitor 12 uses the integrated power spectrum to estimate bearing across a plurality of signal beams, the integrated power spectrum from the range or ranges closest to the target vessel are used by beamformer 48 and/or renal blood flow monitor 12 in the bearing calculation. The separation of range from bearing estimation also simplifies the predictive model used to estimate bearing thereby making the predictive model more robust and reliable.

Similarly, when beamformer 48 and ultrasound transducer probe 14 scans across the field of view to locate the target vessel, the separation of range estimation from bearing estimation advantageously reduces the number of dimensions over which beamformer 48 and ultrasound transducer probe 14 must scan the beam from three dimensions to two dimensions. The estimation methods described in the preceding paragraphs apply equally well to scanning as to tracking.

In order for ultrasound transducer probe 14 to measure the Doppler flow signal DF of the renal blood flow of kidney 42L, ultrasound transducer probe 14 can have a low center frequency between 0.5 MHz and 4.0 MHz. With a center frequency between 0.5 MHz and 4.0 MHz, ultrasound transducer probe 14 can penetrate more than 15 cm into patient 10, which is a sufficient depth to measure the renal blood flow. This depth also allows ultrasound transducer probe 14 the ability to measure hepatic blood flow, celiac blood flow, portal blood flow, and mesenteric blood flow.

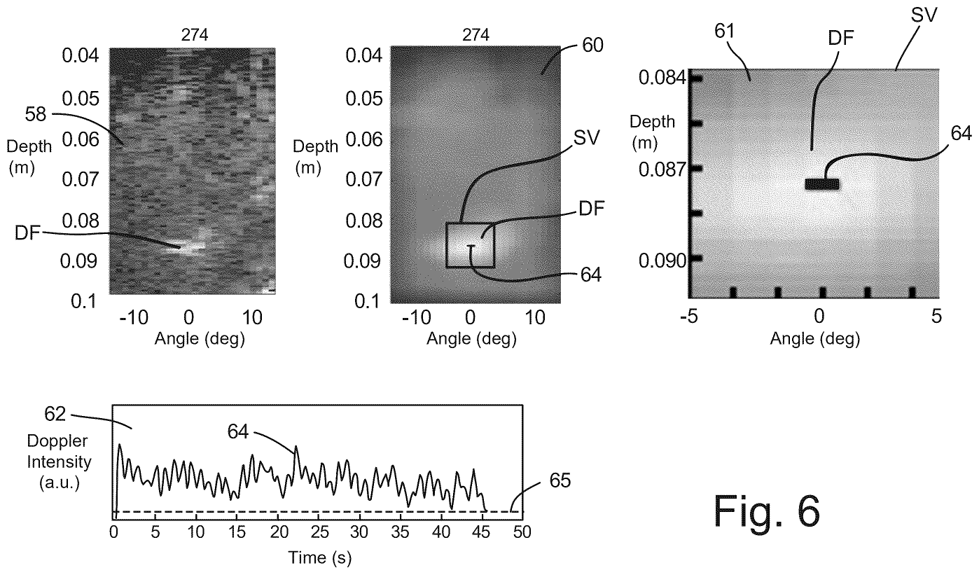

As shown best in the example of FIG. 5, each transducer element 52 in array 50 comprises an element width EW and element length EL that are both larger than one wavelength in soft tissue of an ultrasonic wave emitted by array 50 of transducer elements 52. Array 50 of transducer elements 52 also includes a pitch EP defining an inter-element spacing between centers of adjacent transducer elements 52. In the example of FIG. 5, the pitch EP is larger than the one wavelength in soft tissue of the ultrasonic wave emitted by array 50 of transducer elements 52. The element width EW, the element length EL, and the pitch EP are all larger than the one wavelength in soft tissue of the ultrasonic wave emitted by array 50 of transducer elements 52 to reduce an element count for the selected aperture of ultrasound transducer probe 14. In a traditional phased array imaging transducer, use of a pitch of greater than one wavelength would result in significant image degradation due to grating lobes. However, for ultrasound transducer probe 14, grating lobes do not degrade the Doppler spectrogram because large blood vessels are sparsely distributed in the body and it is highly unlikely that an interfering Doppler signal source would be located at a grating lobe location when a main lobe is focused on a target vessel. Monitoring system 11 does not use ultrasound transducer probe 14 for high resolution imaging of kidney 42L, thus ultrasound transducer probe 14 does not need to have as high a transducer element count as an ultrasound transducer probe used for ultrasound imaging. The way that beamformer 48 and transducer elements 52 in array 50 use signal beams 56 to find and track Doppler flow signal DF in abdomen 40 of patient 10 is discussed in greater detail with reference to FIGS. 6-14.

FIG. 6 discloses first map 58, second map 60, third map 61, and first plot 62 generated by transducer elements 52 in array 50 of ultrasound transducer probe 14. First map 58 is a standard Power Doppler Imaging (PDI) map of a volume of abdomen 40 of patient 10 under a field of view of array 50. As a PDI map, first map 58 shows Doppler intensity of flow signals located within the volume under the field of view of array 50. As shown in FIG. 6, an intensity signature of Doppler flow signal DF of the renal blood flow of kidney 42L appears in a lower portion of first map 58. Second map 60 is a spatially filtered version of first map 58 that provides better resolution of Doppler flow signal DF within the volume. By spatially filtering first map 58, second map 60 better resolves and distinguishes Doppler flow signal DF (brighter shade/color) of the renal blood flow from other pulsatile fluid flows that might be present in the volume. Third map 61 is a spatially filtered PDI map focused and centered on sub-volume SV of the volume from second map 60 that contains a maximum intensity signature 64 of the Doppler flow signal DF. First plot 62 shows a Doppler intensity change in the maximum intensity signature 64 of the Doppler flow signal DF over time.

First map 58, second map 60, and third map 61 are generated by array 50 of ultrasound transducer probe 14 during a finding phase soon after ultrasound transducer probe 14 is positioned onto abdomen 40 of patient 10. The finding phase is when ultrasound transducer probe 14 is locating and identifying the Doppler flow signal DF of the renal blood flow of kidney 42L within abdomen 40. Array 50 of ultrasound transducer probe 14 generates first map 58 by beam scanning the volume under the field of view of array 50 with a set of beams 56. Scanning the volume of abdomen 40 of patient 10 under the field of view of array 50 is first initiated by system processor 18 dividing an entirety of the field of view of array 50 into multiple subsections. Each subsection of the multiple subsections can overlap in area by 25% to 33% with adjacent subsections. Beamformer 48 then drives array 50 to focus a set of beams 56 successively on each subsection of the multiple subsections for a specified amount of time to form first map 58 of the volume. In some examples, the specified amount of time that beams 56 focus on each subsection can be a period of at least one respiration cycle of patient 10. Making the specified amount of time for at least one respiration cycle of patient 10 is one way to take into account movement within the volume of abdomen 40 that is caused by respiration of patient 10. In other examples, the specified amount of time that beams 56 focus on each subsection can be for at least one cardiac cycle of patient 10. In other examples, beamformer 48 can build first map 58 of the volume by using a fast-scanning process. In the fast-scanning process, beamformer 48 drives array 50 to focus for a single beam sequence (˜12 ms) over the entire field of view multiple times over multiple respiration cycles, and then averaging those multiple scans of the entire field of view to build first map 58 of the volume.

System processor 18 spatially filters first map 58 to generate second map 60. As noted above, second map 60 provides a clearer PDI map of the volume under the field of view of array 50 so that system processor 18 and/or an operator can more readily resolve the location of the Doppler flow signal DF of the renal blood flow from any signals in the volume caused by an adjacent conduit and/or vessel in the volume carrying a flow different from the renal blood flow. With first map 58 and second map 60 created, system processor 18 identifies sub-volume SV within the volume that includes the Doppler flow signal DF. To identify the Doppler flow signal DF, system processor 18 searches in the volume for a signature of interest of the Doppler flow signal DF that corresponds to the renal blood flow of patient 10. The signature of interest of the Doppler flow signal DF can be at least one of, or a combination of, but not limited to, signal intensity of the Doppler flow signal DF, signal velocity of the Doppler flow signal DF, spectral shift of the Doppler flow signal DF, signal direction of the Doppler flow signal DF, spectral shift of signals surrounding the Doppler flow signal DF, and signal direction of the signals surrounding the Doppler flow signal DF. In FIG. 6, system processor 18 uses the maximum intensity signature 64 of the Doppler flow signal DF as the signature of interest to identify the renal blood flow of kidney 42L in the volume of second map 60. The maximum intensity signature 64 is the portion of the Doppler flow signal DF in second map 60 that has the highest Doppler intensity. The maximum intensity signature 64 of the Doppler flow signal DF has a Doppler intensity that rises above a preset threshold 65, criterium, criteria, and/or heuristic that is characteristic of renal blood flow. If second plot 60 does not include any Doppler flow signals that rise above the preset threshold 65, criterium, criteria, and/or heuristic that is characteristic of a renal blood flow, system processor 18 can send a signal to display 28 indicating that the renal blood flow of kidney 42L is not within the field of view of the ultrasound transducer probe 14 and that ultrasound transducer probe 14 needs to be repositioned on abdomen 40 of patient 10 and the finding phase repeated.

Once system processor 18 identifies the location of maximum intensity signature 64 in second map 60 of the volume, system processor 18 identifies sub-volume SV by selecting dimensions of sub-volume SV that enclose the maximum intensity signature 64. In one example, system processor 18 can select dimensions for sub-volume SV such that the maximum intensity signature 64 has an intensity decay of 3 dB to 12 dB at edges of sub-volume SV. System processor 18 can output second map 60 with sub-volume SV marked on second map 60 to display 28 so that an operator can verify during the finding phase that system processor 18 has correctly located and identified the Doppler flow signal DF of the renal blood flow of kidney 42L of patient 10. After system processor 18 has identified the location of maximum intensity signature 64 and has defined sub-volume SV that contains the location of maximum intensity signature 64, the finding phase ends and system processor 18 can begin a tracking phase of the Doppler flow signal DF.

In the tracking phase, beamformer 48 drives array 50 to periodically fire a set of beams 56 over sub-volume SV. Beamformer 48 can also direct array 50 to center the set of beams 56 on the location or point of the maximum intensity signature 64. As represented by third map 61, beamformer 48 can direct array 50 to limit scanning during the tracking phase to sub-volume SV such the array 50 is only firing beams 56 at sub-volume SV instead of the whole volume. Limiting scanning to sub-volume SV will increase the SNR, thereby allowing ultrasound transducer probe 14 to get a strong Doppler flow signal DF of the renal blood flow while still tracking the position of the Doppler flow signal DF of the renal blood flow.

If the maximum intensity signature 64 of the Doppler flow signal DF decreases and falls to a level that is below the preset threshold 65, criterium, criteria, and/or heuristic that is characteristic of renal blood flow during the tracking phase, the system processor 18 can stop the tracking phase. The system processor 18, beamformer 48, and array 50 of ultrasound transducer probe 14 will then repeat the finding phase to relocate the Doppler flow signal DF of the renal blood flow and determine a new sub-volume SV that contains the maximum intensity signature 64 of the Doppler flow signal DF. If the system processor 18, beamformer 48, and array 50 do not relocate the Doppler flow signal DF after repeating the finding phase, the system processor 18 will send a signal to display 28 indicating that the renal blood flow of kidney 42L is no longer within the field of view of the ultrasound transducer probe 14, ultrasound transducer probe 14 needs to be repositioned on abdomen 40 of patient 10, and the finding phase repeated.

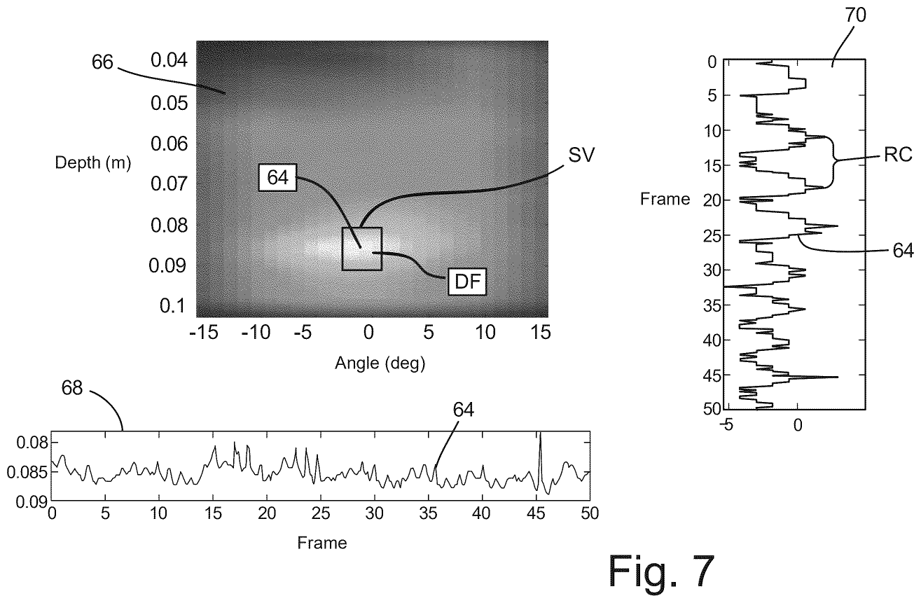

FIG. 7 includes fourth map 66, second plot 68, and third plot 70 generated by transducer elements 52 in array 50 of ultrasound transducer probe 14. Fourth map 66 is an accumulated PDI map of the volume under the field of view of array 50. Second plot 68 shows a depth position of the maximum intensity signature 64 of the Doppler flow signal DF over time in fourth map 66. Third plot 70 shows an angle position of the maximum intensity signature 64 of the Doppler flow signal DF over time in fourth map 66.

Fourth map 66, second plot 68, and third plot 70 can be generated during the finding phase described previously with reference to FIG. 6. During the finding phase, processor 18 divides the entirety of the field of view of array 50 into multiple subsections. Each subsection of the multiple subsections can overlap in area with adjacent subsections to ensure no gaps are inadvertently formed in fourth map 66. For example, each subsection of the multiple subsections can overlap in area by 25% to 33% with adjacent subsections. Beamformer 48 then drives array 50 to focus a set of beams 56 successively on each subsection of the multiple subsections for a period longer than a respiration cycle RC of patient 10 until the set of beams 56 have scanned the entire volume under the field of view of the array 50. The period that beams 56 focus on each subsection is longer than the respiration cycle RC of patient 10 to take into account movement within the volume of abdomen 40 that is caused by respiration RC of patient 10. The entire volume is scanned multiple times by the set of beams 56 until an observation period is completed. In some examples, the observation period spans multiple respiration cycles RC of patient 10. In other examples, the observation period can span multiple cardiac cycles of patient 10. At the end of the observation period, the scans of the volume are combined to form the accumulated PDI map of fourth map 66.

Fourth map 66 has a higher resolution of the volume under the field of view of array 50 than second map 60, which aids system processor 18 in identifying the signature of interest of the Doppler flow signal DF of the renal blood flow of kidney 42L. To identify the Doppler flow signal DF in fourth map 66, system processor 18 searches fourth map 66 of the volume for the signature of interest of the Doppler flow signal DF that corresponds to the renal blood flow of patient 10. The signature of interest of the Doppler flow signal DF can be at least one of signal intensity of the Doppler flow signal DF, signal velocity of the Doppler flow signal DF, spectral shift of the Doppler flow signal DF, signal direction of the Doppler flow signal DF, spectral shift of signals surrounding the Doppler flow signal DF, and signal direction of the signals surrounding the Doppler flow signal DF. Similar to the discussion of FIG. 6, system processor 18 uses the maximum intensity signature 64 of the Doppler flow signal DF as the signature of interest in fourth map 66 of FIG. 7. The maximum intensity signature 64 is the portion of the Doppler flow signal DF in fourth map 66 that has the highest accumulated Doppler intensity over the observation period in fourth map 66.

Once system processor 18 identifies the location of the maximum intensity signature 64 in the volume of fourth map 66, system processor 18 identifies sub-volume SV by selecting dimensions of sub-volume SV that enclose the maximum intensity signature 64 such that the maximum intensity signature 64 has an intensity decay of 3 dB to 12 dB at edges of sub-volume SV. System processor 18 can output fourth map 66 with sub-volume SV marked on fourth map 66 to display 28 so that an operator can verify during the finding phase that system processor 18 has correctly located and identified the Doppler flow signal DF of the renal blood flow of kidney 42L of patient 10.

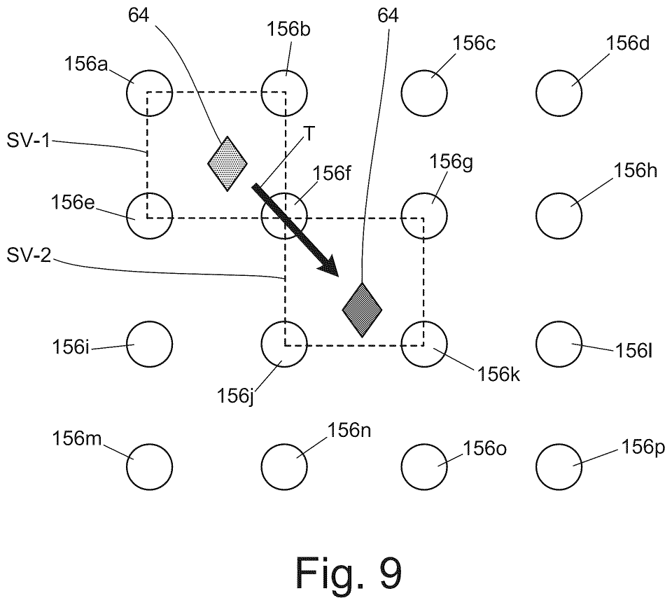

After system processor 18 has identified the location of the maximum intensity signature 64 and sub-volume SV, system processor 18 generates second plot 68 and third plot 70 by using accumulated location information of the maximum intensity signature 64 in fourth map 66. Second plot 68 shows how the depth position of the maximum intensity signature 64 of the Doppler flow signal DF varied over the observation period in fourth map 66. Third plot 70 shows how the angle position of the maximum intensity signature 64 of the Doppler flow signal DF varied over time in fourth map 66. As the finding phase ends and the tracking phase begins, system processor 18 can use second plot 68 and third plot 70 to determine if the position of the maximum intensity signature 64 of the Doppler flow signal DF is moving within the sub-volume SV on a trajectory that will cause the maximum intensity signature 64 to exit sub-volume SV. As discussed below with reference to FIG. 9, system processor 18 during the tracking phase can update the location of sub-volume SV to follow the position of the maximum intensity signature 64, and beamformer 48 can drive transducer elements 52 in array 50 to keep the set of beams 56 focused on sub-volume SV.

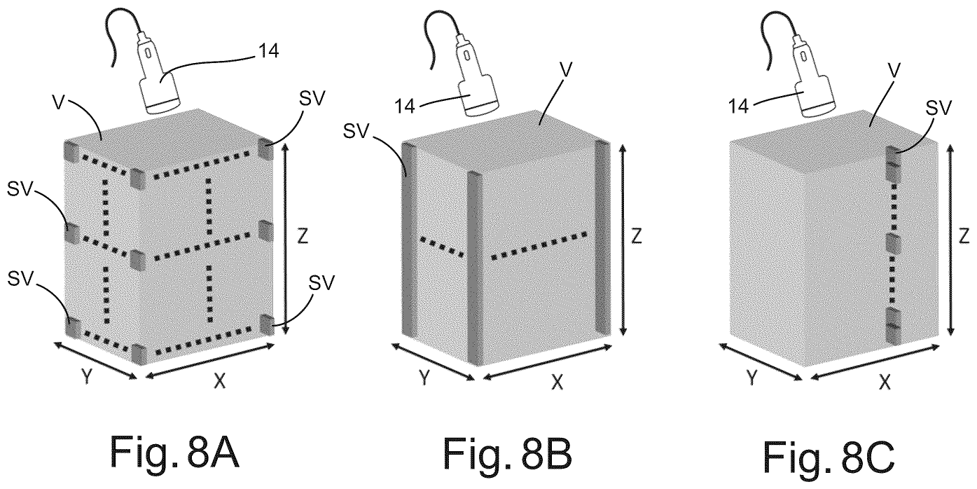

FIG. 8A is a schematic diagram of ultrasound transducer probe 14 scanning volume V of abdomen 40 of patient 10 as described above with reference to FIGS. 6 and 7. The method described in FIGS. 6 and 7 to search for the signal of interest (e.g., the Doppler flow signal DF of the renal blood flow of kidney 42L) can take a significant amount of time to form first map 58, second map 60, third map 61, and/or fourth map 66. This is due to the large number of sub-volumes SV that need to be measured in a three-dimensional field of view. For example, as shown in FIG. 8A, ultrasound transducer probe 14 will scan volume V in first dimension X, second dimension Y, and third dimension Z using small sub-volumes. Furthermore, as the duration of each measurement is increased to capture a cardiac or a respiratory cycle, the search time gets exacerbated. To reduce the search time, the search can be performed in two steps, as discussed below with reference to FIGS. 8B and 8C.

FIGS. 8B and 8C will be discussed concurrently. FIG. 8B is a schematic diagram of ultrasound transducer probe 14 performing a first step S1 for searching volume V for the Doppler flow signal DF in the X and Y dimensions using larger sub-volumes in the Z dimension. FIG. 8C is a schematic diagram of ultrasound transducer probe 14 performing a second step S2 for searching volume V for the Doppler flow signal DF in the Z dimension using smaller sub-volumes. In some examples, first dimension X can be an azimuth dimension extending circumferentially around abdomen 40, second dimension Y is an elevation dimension that extends along a length of abdomen 40, and third dimension Z is a depth dimension extending into abdomen 40 transversely to the first dimension X and the second dimension Y. In the first step S1, ultrasound transducer probe 14 only scans volume V in first dimension X and in second dimension Y. Third dimension Z is not scanned during the first step S1. Since ultrasound transducer probe 14 only scans volume V in first dimension X and second dimension Y during the first step S1, the sub-volume can be larger in third dimension Z than in first and second dimensions. Once ultrasound transducer probe 14 determines a location of the Doppler flow signal DF in first dimension X and second dimension Y, ultrasound transducer probe 14 can move to the second step S2. In the second step S2, ultrasound transducer probe 14 performs a search along third dimension Z at the location of the Doppler flow signal DF in first dimension X and second dimension Y previously determined during the first step S1. By performing a search along third dimension Z at the location of the Doppler flow signal DF in first dimension X and second dimension Y previously determined during the first step S1, ultrasound transducer probe 14 can determine a position of the Doppler flow signal DF in third dimension Z, which completes the search of the Doppler flow signal DF in third volume V3.

Using the approach of the first step S1 and the second step S2 of FIGS. 8B and 8C significantly reduces the search time for the Doppler flow signal DF over the method described in FIGS. 6-8A. To demonstrate the improvement in the search time that can be achieved with the method of FIGS. 8B and 8C, assume a volume under the field of view of array 50 with a size of 3 cm by 2 cm by 5 cm in the X, Y, and Z dimensions respectively. The volume is divided into 240 sub-volumes (6 sub-volumes by 4 sub-volumes by 10 sub-volumes in the X, Y, and Z dimensions respectively), with each sub-volume having a size of 0.5 cm by 0.5 cm by 0.5 cm in the X, Y, and Z dimensions respectively. Under the approach of FIGS. 6-8A, array 50 of ultrasound transducer probe 14 must scan each of the 240 sub-volumes in order to generate a full map of the volume under the field of view of array 50 and to determine a location of the Doppler flow signal DF within the volume.

However, with the two-step search approach of FIGS. 8B and 8C, array 50 of ultrasound transducer probe 14 need only scan 34 sub-volumes (6 sub-volumes by 4 sub-volumes plus 10 sub-volumes in the X, Y, and Z dimensions respectively) out of the 240 sub-volumes to determine a location of the Doppler flow signal DF within the volume. The two-step search approach of FIGS. 8B and 8C is almost an order of magnitude faster than the approach of FIGS. 6-8A. Furthermore, this improvement will only be more evident with larger volumes and/or smaller sub-volumes. For the case of pulsed Doppler ultrasound, the dimension of the sub-volume that can readably be adjusted is third dimension Z (i.e., the depth dimension). This is achieved by changing the duration of the pulses that drive transducer elements 52 of array 50. Alternatively, instead of performing a scan along third dimension Z with different firing beams to find the location of the Doppler flow signal DF of the renal blood flow of kidney 42L, the Doppler signal at all depths can be extracted from the received signal from a single firing beam by adjusting the time delay of the single firing beam.

FIG. 9 is a schematic diagram showing the relative location of beams 156a-156p used for tracking the maximum intensity signature 64 of the Doppler flow signal DF of the renal blood flow of kidney 42L as the renal blood flow shifts positions within the field of view of array 50. FIG. 9 shows the maximum intensity signature 64 being positioned in first sub-volume SV-1. FIG. 9 also shows a predicted position trajectory T of the maximum intensity signature 64 and second sub-volume SV-2 that the maximum intensity signature 64 is predicted to enter.

Beams 156a-156p are precalculated beams that are stored in system memory 20. Beams 156a-156p can be precalculated by system processor 18 during the finding phase, or beams 156a-156p can be precalculated and saved to system memory 20 earlier than the finding phase. A precalculated table (not shown) in system memory 20 spatially links beams 156a-156p with the volume under the field of view of array 50 such that an individual coverage area or volume for each of beams 156a-156p is predetermined and known by system processor 18. The precalculated table can also link each of beams 156a-156p into predetermined beam sets that are linked to a specific coverage area of the volume under the field of view of array 50. The precalculated table can also link a predetermined beam set with adjacent beam sets. Having beams 156a-156p precalculated and linked to the volume under the field of view of array 50 by the precalculated table allows system processor 18 and beamformer 48 to quickly drive array 50 and steer tracking of the position of the maximum intensity signature 64 of the Doppler flow signal DF.

At the beginning of the tracking phase, in the example of FIG. 9, the maximum intensity signature 64 of the Doppler flow signal DF is positioned within first sub-volume SV-1. Using the precalculated information regarding beams 156a-156p and the precalculated table, system processor 18 determines that beams 156a, 156b, 156e, and 156f can form a beam set that will cover first sub-volume SV-1. Through beamformer 48, system processor 18 drives array 50 of ultrasound transducer probe 14 to generate beams 156a, 156b, 156e, and 156f to monitor first sub-volume SV-1 for as long as the maximum intensity signature 64 of the Doppler flow signal DF is positioned within first sub-volume SV-1. To increase the SNR of the Doppler flow signal DF, array 50 only fires beams 156a, 156b, 156e, and 156f while the maximum intensity signature 64 is positioned within first sub-volume SV-1. Firing all of beams 156a-156p all the time would increase noise and reduce the SNR of the Doppler flow signal DF as well as reduce the bandwidth of monitoring system 11.

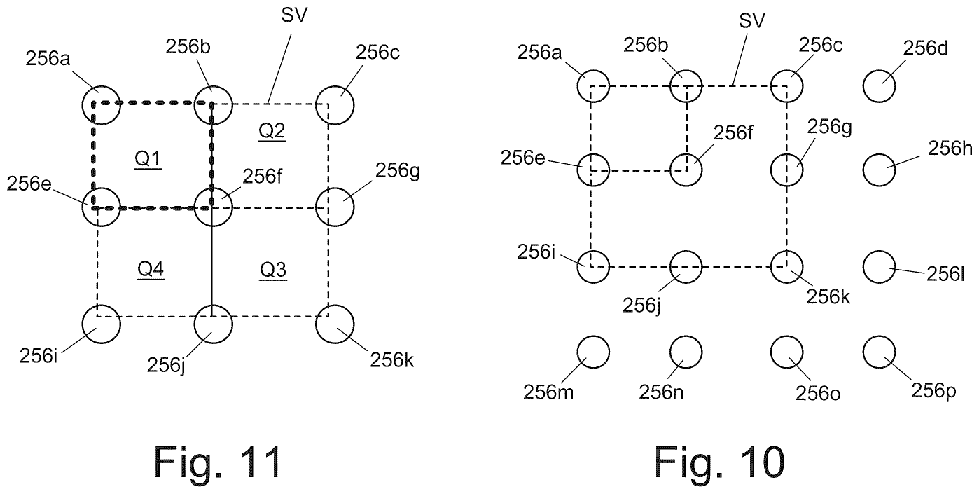

If system processor 18 determines that the maximum intensity signature 64 is moving on the predicted position trajectory T, system processor 18 can detect when the position of the maximum intensity signature 64 is at an edge of first sub-volume SV-1 and an edge of the combined coverage area of beams 156a, 156b, 156e, and 156f. To maintain tracking and monitoring of the maximum intensity signature 64 of the Doppler flow signal DF, system processor 18 can determine second sub-volume SV-2 that the maximum intensity signature 64 is predicted to enter. Once system processor 18 has determined second sub-volume SV-2, system processor 18 can use the precalculated information regarding beams 156a-156p and the precalculated table to determine that beams 156f, 156g, 156j, and 156k can form a beam set that will cover second sub-volume SV-2. Through beamformer 48, system processor 18 drives array 50 to generate beams 156f, 156g, 156j, and 156k to monitor second sub-volume SV-2 just before the maximum intensity signature 64 enters second sub-volume SV-2. After the maximum intensity signature 64 of the Doppler flow signal DF enters second sub-volume SV-2 and exits first sub-volume SV-1, beamformer 48 directs array 50 to cease firing beams 156a, 156b, and 156e, such that array 50 is only firing beams 156f, 156g, 156j, and 156k. System processor 18 drives array 50 of ultrasound transducer probe 14 to generate beams 156f, 156g, 156j, and 156k to monitor second sub-volume SV-2 for as long as the maximum intensity signature 64 of the Doppler flow signal DF is positioned within second sub-volume SV-2. Furthermore, to make the signal more stable in the depth dimension and to improve signal tracking, the sub-volumes SV can be made larger in the depth dimension by increasing the duration of the driving pulses used to form the firing beams or by extracting the Doppler signal at all depths from the received signal from a single firing beam by adjusting the time delay and selecting the one with the highest intensity or an average of the stronger ones. As discussed below with reference to FIGS. 10-14, multiple arrangements and configurations of precalculated beams can be used by system processor 18, beamformer 48, and array 50 of ultrasound transducer probe 14 to track and monitor the maximum intensity signature 64 (or any other signature of interest) of the Doppler flow signal DF of the renal blood flow of patient 10.

FIGS. 10 and 11 will be discussed concurrently. FIG. 10 is a schematic diagram showing the relative location of beams 256a-256p and FIG. 11 is a schematic diagram of a beam set selected from beams 256a-256p of FIG. 10. Similar to beams 156a-156p of FIG. 9, beams 256a-256p of FIG. 10 are precalculated beams that are stored in system memory 20. Beams 256a-256p can be precalculated by system processor 18 during the finding phase, or beams 256a-256p can be precalculated and saved to system memory 20 earlier than the finding phase. A precalculated table (not shown) in system memory 20 spatially links beams 256a-256p with the volume under the field of view of array 50 such that an individual coverage area or volume for each of beams 256a-256p is predetermined and known by system processor 18. The precalculated table can also link each of beams 256a-256p into predetermined beam sets that are linked to a specific coverage area of the volume under the field of view of array 50. The precalculated table can also link a predetermined beam set with adjacent beam sets. Having beams 256a-256p precalculated and linked to the volume under the field of view of array 50 by the precalculated table allows system processor 18 and beamformer 48 to quickly drive array 50 and steer tracking of the position of the maximum intensity signature 64 of the Doppler flow signal DF.

In FIGS. 10 and 11, each precalculated beam set includes an assigned central beam with the rest of the beams in the beam set surrounding the central beam. During the tracking phase, beamformer 48 drives array 50 to direct and periodically fire the central beam on the location or point of the maximum intensity signature 64 of the Doppler flow signal DF. The beams in the beam set that surround the central beam are directed at sub-volume SV that contains the location or point of the maximum intensity signature 64 of the Doppler flow signal DF. The central beam is fired more frequently by array 50 than the surrounding beams to increase the SNR of the Doppler flow signal DF.

For example, in FIGS. 10 and 11, beams 256a, 256b, 256c, 256e, 256f, 256g, 256i, 256j, and 256k form a beam set for tracking and monitoring sub-volume SV. Beam 256f is assigned as the central beam of this beam set while beams 256a, 256b, 256c, 256e, 256g, 256i, 256j, and 256k are assigned as surrounding beams in the beam set. Sub-volume SV is generally centered over the maximum intensity signature 64 of the Doppler flow signal DF. While tracking and monitoring sub-volume SV, center beam 256f is firing at a higher rate than beams 256a, 256b, 256c, 256e, 256g, 256i, 256j, and 256k to generate a clear reading of the Doppler flow signal DF of the renal blood flow for health monitoring. Surrounding beams 256a, 256b, 256c, 256e, 256g, 256i, 256j, and 256k only need to fire at a rate that is sufficient to track the position of the maximum intensity signature 64 of the Doppler flow signal DF inside sub-volume SV. An intensity of each of beams 256a, 256b, 256c, 256e, 256f, 256g, 256i, 256j, and 256k at a maximum intersection point between adjacent beams of the beam set is 3 dB to 6 dB to minimize interference between beams and increase the SNR of the Doppler flow signal DF. System processor 18 can also utilize a weighted average sum of beams 256a, 256b, 256c, 256e, 256f, 256g, 256i, 256j, and 256k to maximize the SNR of the Doppler flow signal DF.

To further improve the SNR, system processor 18 can divide sub-volume SV into quadrants Q1-Q4, and array 50 can focus scanning on a single quadrant at a time while cycling through all of quadrants Q1-Q4. For example, as shown in FIG. 11, array 50 can only fire beams 256a, 256b, 256e, and 256f to scan first quadrant Q1 of sub-volume SV. Next, array 50 can only fire beams 256b, 256c, 256g, and 256f to scan second quadrant Q2 of sub-volume SV. Next, array 50 can only fire beams 256g, 256k, 256j, and 256f to scan third quadrant Q3 of sub-volume SV. Next, array 50 can only fire beams 256e, 256i, 256j, and 256f to scan fourth quadrant Q4 of sub-volume SV. After scanning fourth quadrant Q4, array 50 can repeat the process. Since center beam 256f is included in the scanning of every quadrant Q1-Q4, center beam 256f fires at a higher rate than the rest of the beams monitoring sub-volume SV. If the position of the maximum intensity signature 64 of the Doppler flow signal DF shifts out of alignment with center beam 256f such that the position of the maximum intensity signature 64 is still within sub-volume SV but is between beams, system processor 18 can estimate the position of the maximum intensity signature 64 within sub-volume SV by calculating a centroid or mean of the Doppler flow signal DF from each of beams 256a, 256b, 256c, 256e, 256f, 256g, 256i, 256j, and 256k in the beam set.

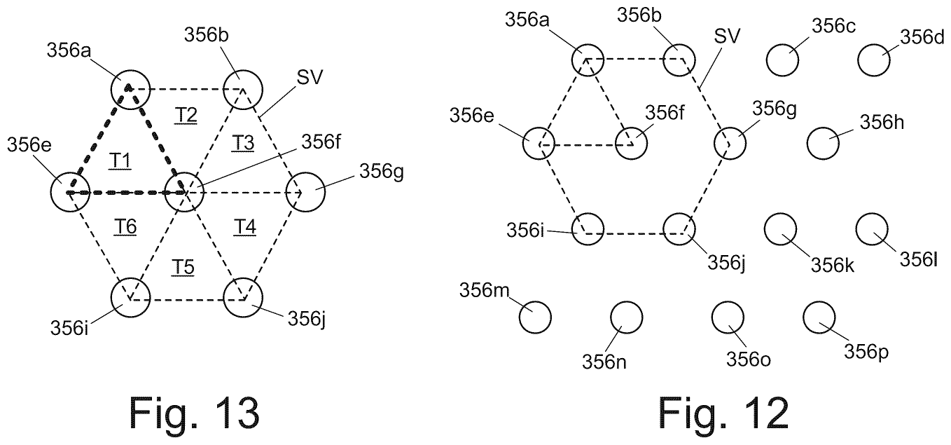

FIGS. 12 and 13 will be discussed concurrently. FIG. 12 is a schematic diagram showing the relative location of beams 356a-356p. FIG. 13 is a schematic diagram of a beam set that includes beams 356a, 356b, 356e, 356f, 356g, 356i, and 356j from FIG. 12. Similar to beams 256a-256p of FIG. 10, beams 356a-356p of FIG. 12 are precalculated beams that are stored in system memory 20. A precalculated table (not shown) in system memory 20 spatially links beams 356a-356p with the volume under the field of view of array 50 such that an individual coverage area or volume for each of beams 356a-356p is predetermined and known by system processor 18. The precalculated table can also link each of beams 356a-356p into predetermined beam sets that are linked to a specific coverage area of the volume under the field of view of array 50. The precalculated table can also link a predetermined beam set with adjacent beam sets.

Similar to the example of FIGS. 10 and 11, each precalculated beam set for beams 356a-356p of FIG. 12 includes an assigned central beam with the rest of the beams in the beam set surrounding the central beam. In FIGS. 12 and 13, beams 356a-356p are arranged in offset rows that allow system processor 18 to form sub-volume SV based on a grouping of triangles T1-T6 arranged around a center beam.

As shown in FIG. 13, sub-volume SV is divided into triangles T1-T6. During the tracking phase, array 50 only fires beams 356a, 356e, and 356f to scan first triangle T1 of sub-volume SV. Furthermore, to make the signal more stable in the depth dimension and to improve signal tracking, the sub-volumes SV can be made larger in the depth dimension by increasing the duration of the driving pulses used to form the firing beams or by extracting the Doppler signal at all depths from the received signal from a single firing beam by adjusting the time delay and selecting the one with the highest intensity or an average of the stronger ones. Next, array 50 only fires beams 356a, 356b, and 356f to scan second triangle T2 of sub-volume SV. After scanning second triangle T2, array 50 only fires beams 356b, 356g, and 356f to scan third triangle T3 of sub-volume SV. Next, array 50 only fires beams 356g, 356j, and 356f to scan fourth triangle T4 of sub-volume SV. After scanning fourth triangle T4, array 50 only fires beams 356j, 356i, and 356f to scan fifth triangle T5 of sub-volume SV. Next, array 50 only fires beams 356i, 356e, and 356f to scan sixth triangle T6 of sub-volume SV.

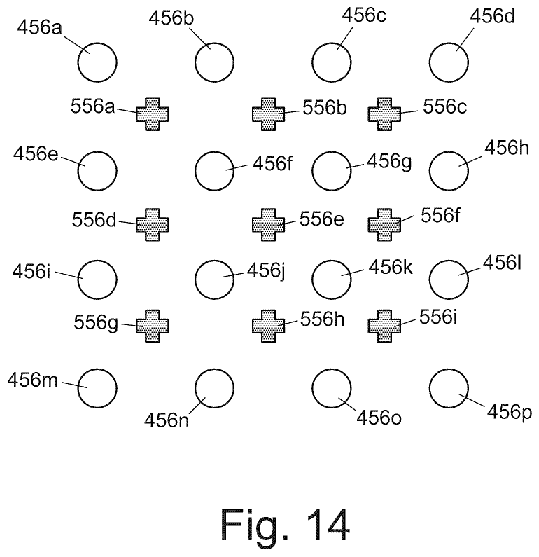

After scanning sixth triangle T6, array 50 can repeat the process. Since center beam 356f is included in the scanning of every triangle T1-T6, center beam 356f fires at a higher rate than the rest of the beams monitoring sub-volume SV in FIG. 12. If the position of the maximum intensity signature 64 of the Doppler flow signal DF shifts out of alignment with center beam 356f such that the position of the maximum intensity signature 64 is still within sub-volume SV but is between beams, system processor 18 can estimate the position of the maximum intensity signature 64 within sub-volume SV by calculating a centroid or mean of the Doppler flow signal DF from each of beams 356a, 356b, 356e, 356f, 356g, 356i, and 356j in the beam set. In addition to using precalculated beams and beam sets, system processor 18 can use synthetic focusing to produce synthetic beams to track and monitor the maximum intensity signature 64 (or any other signature of interest) of the Doppler flow signal DF of the renal blood flow of patient 10, as discussed below with reference to FIG. 14.

FIG. 14 is a schematic diagram of a beam set that includes beams 456a-456p and synthetic beams 556a-556i. Similar to beams 256a-256p of FIG. 10, beams 456a-456p of FIG. 14 are precalculated beams that are stored in system memory 20 and used by beamformer 48 and array 50 to create real send and/or receive ultrasound beams. Synthetic beams 556a-556i are virtual beams computed by system processor 18. System processor 18 uses data received by transducer elements 52 of array 50 and then manipulates the data to artificially produce synthetic beams 556a-556i. System processor 18 can use phase delays, time delays, and/or additional synthetic focusing techniques to generate synthetic beams 556a-556i. By using synthetic beams 556a-556i in addition to beams 456a-456p, monitoring system 11 can increase the resolution of the beam scanning and tracking of ultrasound transducer probe 14.

### Discussion of Possible Embodiments

The following are non-exclusive descriptions of possible embodiments of the present invention.

A method for monitoring renal blood flow of a patient is disclosed. The method includes affixing an ultrasound transducer probe on an abdomen of the patient. The ultrasound transducer probe includes a two-dimensional array of transducer elements. The two-dimensional array of transducer elements and a beamformer driving the two-dimensional array of transducer elements scan a volume of the abdomen of the patient under a field of view of the two-dimensional array of transducer elements. A processor in communication with the ultrasound transducer probe and the beamformer identifies a sub-volume in the volume with a Doppler flow signal comprising a signature of interest corresponding to the renal blood flow of the patient. Periodically firing a sequential set of beams from the two-dimensional array of transducer elements over the sub-volume to track the sub-volume.

The method of the preceding paragraph can optionally include, additionally and/or alternatively, any one or more of the following features, configurations and/or additional components in the paragraphs below.

In an embodiment of the foregoing method, wherein scanning, by the two-dimensional array of transducer elements and the beamformer driving the two-dimensional array of transducer elements, the volume of the abdomen of the patient under the field of view of the two-dimensional array of transducer elements comprises: scanning, by the two-dimensional array of transducer elements and the beamformer driving the two-dimensional array of transducer elements, the volume in a first dimension and in a second dimension only to determine a location of the renal blood flow of the patient in the first dimension and the second dimension; and scanning, by the two-dimensional array of transducer elements and the beamformer driving the two-dimensional array of transducer elements, the volume in a third dimension only at the location of the renal blood flow of the patient in the first dimension and the second dimension to determine a location of the renal blood flow of the patient in the third dimension.

In an embodiment of the foregoing method, the third dimension is a depth dimension extending into the abdomen of the patient.

In an embodiment of the foregoing method, the two-dimensional array of transducer elements scans the volume in the third dimension more frequently than first dimension and the second dimension.

In an embodiment of the foregoing method, the method further comprises: detecting, by the processor in communication with the ultrasound transducer probe and the beamformer, when a location of maximum intensity of the signature of interest is at an edge of the sequential set of beams and/or has a trajectory that is shifting the location of the maximum intensity of the signature of interest toward the edge of the sequential set of beams; and steering the sequential set of beams, by the beamformer driving the two-dimensional array of transducer elements, to maintain the location of the maximum intensity of the signature of interest at a center of the sequential set of beams.

In an embodiment of the foregoing method, the method further comprises steering the sequential set of beams to a location based on a predicted future location of the maximum intensity of the signature of interest.

In an embodiment of the foregoing method, the method further comprises: detecting, by the processor in communication with the ultrasound transducer probe and the beamformer, when a location of maximum intensity of the signature of interest is at an edge of the sequential set of beams and/or has a trajectory that is shifting the location of the maximum intensity of the signature of interest toward the edge of the sequential set of beams; and steering the sequential set of beams, by the beamformer driving the two-dimensional array of transducer elements, to maintain the location of the maximum intensity of the signature of interest at a center of the sequential set of beams.

In an embodiment of the foregoing method, the method further comprises steering the sequential set of beams to a location based on a predicted future location of the maximum intensity of the signature of interest.

In an embodiment of the foregoing method, steering the sequential set of beams to the location based on the predicted future location of the maximum intensity of the signature of interest comprises: measuring estimates of a location of the renal blood flow by the beamformer; inputting the estimates of the location of the renal blood flow into a predictive filter; and determining, by the processor in communication with the ultrasound transducer probe and the beamformer, an expected trajectory of the location of the renal blood flow based on the estimates of the location of the renal blood flow and based on a breathing frequency of the patient.

In an embodiment of the foregoing method, measuring estimates of the location of the renal blood flow by the beamformer comprises: measuring, by the beamformer, differences in integrated power spectrum signal between individual beams of the set of sequential beams to estimate an azimuthal angle and an elevation angle of the location of the renal blood flow relative to the array of transducer elements; and estimating, by the beamformer, a distance of the blood flow from the array of transducer elements in a distance dimension by: gathering, by the array of transducer elements and the beamformer, a plurality of distance samples along a distance dimension; calculating, by the beamformer, integrated power spectrum signal for each distance sample of the plurality of distance samples; assigning, by the beamformer, a likelihood of containing the renal blood flow to each distance sample of the plurality of distance samples; and calculating, by the beamformer, an estimate of a center of the renal blood flow from the plurality of distance samples.

In an embodiment of the foregoing method, the method further comprises: making, by the beamformer, proportional the likelihood of containing the renal blood flow to the integrated power spectrum signal for each distance sample of the plurality of distance samples; and calculating, by the beamformer, the estimate of the center of the renal blood flow from the plurality of distance samples by selecting a distance sample of the plurality of distance samples with the largest integrated power spectrum signal.

In an embodiment of the foregoing method, the method further comprises: measuring the breathing frequency of the patient with a breathing monitor connected to the patient; and inputting the breathing frequency of the patient into the predictive filter from the breathing monitor.

In an embodiment of the foregoing method, the method further comprises measuring, by the beamformer, the breathing frequency of the patient by halting steering of the sequential set of beams for a period of time such that a position or positions of the sequential set of beams in the first dimension and the second dimension are fixed during the period of time; observing, by the beamformer, a periodicity of integrated power spectrum signal of the sequential set of beams while the steering of the sequential set of beams is halted; estimating the breathing frequency from the periodicity of the integrated power spectrum signal of the sequential set of beams while the steering of the sequential set of beams is halted; and inputting, by the beamformer, the estimated breathing frequency of the patient into the predictive filter.

In an embodiment of the foregoing method, the method further comprises: assigning over time, by the beamformer and/or the processor, an uncertainty estimate to new measurements of the estimates of the location of the renal blood flow based on a presence of a transient event, a signal-to-noise ratio of the integrated power spectrum signal, changes in moments of instantaneous spectrum of a Doppler flow signal of the renal blood flow, or changes in a maximum velocity envelope of a Doppler spectrogram of the renal blood flow; and continuously inputting, from the beamformer and/or the processor to the predictive filter, the uncertainty estimate to adjust a degree or weight to which model parameters of the predictive filter are affected by new measurements during model parameter updates based upon the uncertainty estimate, such that the predictive filter can ignore measurements that are invalid.

In an embodiment of the foregoing method, the method further comprises: determining, by the predictive filter, an estimate of the integrated power spectrum signal along each beam of the beam set based on a physical model of the location of the renal blood flow; comparing, by the predictive filter, the estimate of the integrated power spectrum signal along each beam of the beam set to measurements of the integrated power spectrum signal along each signal beam of the beam set respectively; and updating, by the processor in communication with the ultrasound transducer probe and the beamformer, model parameters of the physical model of the location of the renal blood flow based on differences between the estimate of the integrated power spectrum signal and the measurements of the integrated power spectrum signal along each signal beam of the beam set, wherein the model parameters of the physical model comprises beam shape of each beam of the beam set, vessel shape and orientation of a vessel containing the renal blood flow, and location of the vessel within the patient.

In an embodiment of the foregoing method, determining, by the predictive filter, the estimate of the integrated power spectrum signal along each beam of the beam set based on the physical model of the location of the renal blood flow comprises: calculating, by the physical model an overlap integral between the vessel and an ultrasound beam profile of each beam of the beam set; and wherein the model parameters of the physical model comprise a description of how the ultrasound beam profile of each beam of the beam set changes with depth.

In an embodiment of the foregoing method, the predictive filter comprises a Kalman Filter.

In an embodiment of the foregoing method, the sequential set of beams comprises a central beam directed to the location of the maximum intensity of the signature of interest by the beamformer, and wherein the sequential set of beams comprises surrounding beams directed at the sub-volume by the beamformer that surround the central beam.

In an embodiment of the foregoing method, an intensity of each beam of the sequential set of beams at a maximum intersection point between adjacent beams of the beam set is 3 dB to 6 dB.

In an embodiment of the foregoing method, the central beam is fired more frequently than the surrounding beams to increase a signal-to-noise ratio (SNR) of the measured Doppler flow signal.

In an embodiment of the foregoing method, the signature of interest of the Doppler flow signal comprises at least one of signal intensity of the Doppler flow signal, signal velocity of the Doppler flow signal, spectral shift of the Doppler flow signal, signal direction of the Doppler flow signal, spectral shift of signals surrounding the Doppler flow signal, and signal direction of the signals surrounding the Doppler flow signal.

In an embodiment of the foregoing method, the method further comprises limiting, by the beamformer, scanning of the two-dimensional array of transducer elements to the sub-volume while tracking the sub-volume by periodically firing the sequential set of beams from the two-dimensional array of transducer elements over the sub-volume.

In an embodiment of the foregoing method, the method further comprises defining, by the processor, dimensions of the sub-volume to contain the signature of interest of the Doppler flow signal, wherein the signature of interest has an intensity decay of 3 dB to 12 dB at edges of the sub-volume.

In an embodiment of the foregoing method, scanning, by the two-dimensional array of transducer elements and the beamformer driving the two-dimensional array of transducer elements, the volume of the abdomen of the patient under the field of view of the two-dimensional array of transducer elements comprises: dividing, by the processor, an entirety of the field of view of the two-dimensional array of transducer elements into multiple subsections, wherein each subsection of the multiple subsections overlaps in area by 25% to 33% with adjacent subsections; focusing, by the two-dimensional array of transducer elements and the beamformer driving the two-dimensional array of transducer elements, multiple beams on each subsection of the multiple subsections for a period to form a Power Doppler Imaging (PDI) map of the volume; and repeating in succession the focusing of the multiple beams on each subsection of the multiple subsections over an observation period to generate an accumulated PDI map of the volume.

In an embodiment of the foregoing method, the period is at least one respiration cycle of the patient, and the observation period spans multiple respiration cycles of the patient.

In an embodiment of the foregoing method, the period is at least one cardiac cycle of the patient, and the observation period spans multiple cardiac cycles of the patient.

In an embodiment of the foregoing method, scanning, by the two-dimensional array of transducer elements and the beamformer driving the two-dimensional array of transducer elements, the volume of the abdomen of the patient under the field of view of the two-dimensional array of transducer elements comprises: scanning, by the two-dimensional array of transducer elements and the beamformer driving the two-dimensional array of transducer elements, an entirety of the field of view of the two-dimensional array of transducer elements for multiple times over multiple respiration cycles of the patient to form a Power Doppler Imaging (PDI) map of the volume.

In an embodiment of the foregoing method, identifying, by the processor in communication with the ultrasound transducer probe and the beamformer, the sub-volume in the volume with the Doppler flow signal comprising the signature of interest corresponding to the renal blood flow of the patient comprises: selecting the subsection of the multiple subsections with a highest accumulated intensity over the observation period in the accumulated PDI map as the sub-volume in the volume with the Doppler flow signal comprising the signature of interest corresponding to the renal blood flow of the patient.

In an embodiment of the foregoing method, the method further comprises: spatially filtering the sub-volume in the PDI map and/or in the accumulated PDI map to resolve the renal blood flow from an adjacent conduit and/or vessel carrying a flow different from the renal blood flow; selecting a point of maximum intensity of the signature of interest from the spatially filtered sub-volume; and centering the sequential set of beams on the point of maximum intensity of the signature of interest.

In an embodiment of the foregoing method, the method further comprises: rescanning, by the two-dimensional array of transducer elements and the beamformer driving the two-dimensional array of transducer elements, the volume of the abdomen of the patient under the field of view of the two-dimensional array of transducer elements if the signature of interest of the Doppler flow signal falls below a preset threshold, criterium, criteria, and/or heuristic while tracking the sub-volume; and reidentifying, by the processor in communication with the ultrasound transducer probe and the beamformer, the sub-volume in the volume with the Doppler flow signal comprising the signature of interest corresponding to the renal blood flow of the patient.

In an embodiment of the foregoing method, the method further comprises sending a signal, by the processor, to a display indicating that the signature of interest cannot be found and that the ultrasound transducer probe needs to be repositioned on the abdomen of the patient when the processor cannot identify in the volume the Doppler flow signal comprising the signature of interest above the preset threshold, criterium, criteria, and/or heuristic.

In an embodiment of the foregoing method, the method further comprises estimating, by the processor, a location of the Doppler flow signal comprising the signature of interest in the volume by calculating a centroid or mean of the Doppler flow signal comprising the signature of interest from each beam in the sequential set of beams.

In an embodiment of the foregoing method, the method further comprises utilizing, by the processor, a weighted average sum of beams in the sequential set of beams to maximize a signal-to-noise ratio (SNR) of the Doppler flow signal.

In an embodiment of the foregoing method, beams in the sequential set of beams overlap with one another.

In an embodiment of the foregoing method, the sequential set of beams is precalculated and stored in system memory in communication with the beam former and the processor, wherein a precalculated table in the system memory links the sequential set of beams with a coverage area of the sequential set of beams, and wherein the precalculated table links the sequential set of beams with a neighboring beam and/or a neighboring beam set and a coverage area of the neighboring beam and/or the neighboring beam set.

In an embodiment of the foregoing method, the method further comprises synthetically forming additional beams for the sequential set of beams to increase a granularity and/or signal-to-noise ratio (SNR) of the Doppler flow signal while tracking the sub-volume.

A method is disclosed for monitoring an organ blood flow of a patient. The method includes affixing an ultrasound transducer probe on an abdomen of the patient. The ultrasound transducer probe includes a two-dimensional array of transducer elements. A finding phase is performed by scanning, by the two-dimensional array of transducer elements and a beamformer driving the two-dimensional array of transducer elements, a volume of the abdomen of the patient under the field of view of the two-dimensional array of transducer elements. A processor in communication with the ultrasound transducer probe and the beamformer identifies a sub-volume in the volume with a Doppler flow signal comprising a signature of interest corresponding to the organ blood flow of the patient. A tracking phase of the Doppler flow signal comprising the signature of interest of the organ blood flow is performed by periodically firing a beam set from the two-dimensional array of transducer elements over the sub-volume.

The method of the preceding paragraph can optionally include, additionally and/or alternatively, any one or more of the following features, configurations and/or additional components in the paragraphs below.

In an embodiment of the foregoing method, the method further comprises: detecting, by the processor in communication with the ultrasound transducer probe and the beamformer, when a location of maximum intensity of the signature of interest is at an edge of the beam set and/or has a trajectory that is shifting the location of the maximum intensity of the signature of interest toward the edge of the beam set; and steering the beam set, by the beamformer driving the two-dimensional array of transducer elements, to maintain the location of the maximum intensity of the signature of interest at a center of the beam set.

In an embodiment of the foregoing method, the method further comprises steering the beam set to a location based on a predicted future location of the maximum intensity of the signature of interest.

In an embodiment of the foregoing method, the beam set comprises a central beam directed to the location of the maximum intensity of the signature of interest by the beamformer, and wherein the beam set comprises surrounding beams directed at the sub-volume by the beamformer and that surround the central beam.

In an embodiment of the foregoing method, an intensity of each beam of the beam set at a maximum intersection point between adjacent beams of the beam set is 3 dB to 6 dB.

In an embodiment of the foregoing method, the method further comprises firing the central beam more frequently than the surrounding beams to increase a signal-to-noise ratio (SNR) of the Doppler flow signal.

In an embodiment of the foregoing method, the signature of interest of the Doppler flow signal comprises at least one of signal intensity of the Doppler flow signal, signal velocity of the Doppler flow signal, spectral shift of the Doppler flow signal, signal direction of the Doppler flow signal, spectral shift of signals surrounding the Doppler flow signal, and signal direction of the signals surrounding the Doppler flow signal.

In an embodiment of the foregoing method, the method further comprises limiting, by the beamformer, scanning of the two-dimensional array of transducer elements to the sub-volume during the tracking phase.

In an embodiment of the foregoing method, the method further comprises defining, by the processor, dimensions of the sub-volume to contain the signature of interest of the Doppler flow signal, wherein the signature of interest has an intensity decay of 3 dB to 12 dB at edges of the sub-volume.

In an embodiment of the foregoing method, scanning, by the two-dimensional array of transducer elements and the beamformer driving the two-dimensional array of transducer elements, the volume of the abdomen of the patient under the field of view of the two-dimensional array of transducer elements comprises: dividing, by the processor, an entirety of the field of view of the two-dimensional array of transducer elements into multiple subsections, wherein each subsection of the multiple subsections overlaps in area by 25% to 33% with adjacent subsections; focusing, by the two-dimensional array of transducer elements and the beamformer driving the two-dimensional array of transducer elements, multiple beams on each subsection of the multiple subsections for a period to form a Power Doppler Imaging (PDI) map of the volume; and repeating in succession the focusing of the multiple beams on each subsection of the multiple subsections over an observation period to generate an accumulated PDI map of the volume.

In an embodiment of the foregoing method, the period is at least one respiration cycle of the patient, and the observation period spans multiple respiration cycles of the patient.

In an embodiment of the foregoing method, the period is at least one cardiac cycle of the patient, and the observation period spans multiple cardiac cycles of the patient.

In an embodiment of the foregoing method, scanning, by the two-dimensional array of transducer elements and the beamformer driving the two-dimensional array of transducer elements, the volume of the abdomen of the patient under the field of view of the two-dimensional array of transducer elements comprises: scanning, by the two-dimensional array of transducer elements and the beamformer driving the two-dimensional array of transducer elements, an entirety of the field of view of the two-dimensional array of transducer elements for multiple times over multiple respiration cycles of the patient to form a Power Doppler Imaging (PDI) map of the volume.

In an embodiment of the foregoing method, identifying, by the processor in communication with the ultrasound transducer probe and the beamformer, the sub-volume in the volume with the Doppler flow signal comprising the signature of interest corresponding to the organ blood flow of the patient comprises: selecting the subsection of the multiple subsections with a highest accumulated intensity over the observation period in the accumulated PDI map as the sub-volume in the volume with the Doppler flow signal comprising the signature of interest corresponding to the organ blood flow of the patient.

In an embodiment of the foregoing method, the method further comprises: spatially filtering the sub-volume in the PDI map and/or in the accumulated PDI map to resolve the organ blood flow from an adjacent conduit and/or vessel carrying a flow different from the organ blood flow; selecting a point of maximum intensity of the signature of interest from the spatially filtered sub-volume; and centering the beam set on the point of maximum intensity of the signature of interest.

In an embodiment of the foregoing method, the method further comprises repeating the finding phase if the signature of interest of the Doppler flow signal falls below a preset threshold, criterium, criteria, and/or heuristic while performing the tracking phase.

In an embodiment of the foregoing method, the method further comprises sending a signal, by the processor, to a display indicating that the signature of interest cannot be found and that the ultrasound transducer probe needs to be repositioned on the abdomen of the patient when the processor cannot identify in the volume the Doppler flow signal comprising the signature of interest above the preset threshold, criterium, criteria, and/or heuristic.

In an embodiment of the foregoing method, the method further comprises estimating, by the processor, a location of the Doppler flow signal comprising the signature of interest in the volume by calculating a centroid or mean of the Doppler flow signal comprising the signature of interest from each beam in the beam set.

In an embodiment of the foregoing method, the method further comprises utilizing, by the processor, a weighted average sum of beams in the beam set to maximize a signal-to-noise ratio (SNR) of the Doppler flow signal.

In an embodiment of the foregoing method, beams in the beam set overlap with one another.

In an embodiment of the foregoing method, the beam set is precalculated and stored in system memory in communication with the beam former and the processor, wherein a precalculated table in the system memory links the beam set with a coverage area of the beam set, and wherein the precalculated table links the beam set with a neighboring beam and/or a neighboring beam set and a coverage area of the neighboring beam and/or the neighboring beam set.

In an embodiment of the foregoing method, the method further comprising synthetically forming additional beams for the beam set to increase a granularity and/or signal-to- noise ratio (SNR) of the Doppler flow signal while tracking the sub-volume.

In an embodiment of the foregoing method, the organ blood flow is a renal blood flow of the patient.

While the invention has been described with reference to an exemplary embodiment(s), it will be understood by those skilled in the art that various changes may be made and equivalents may be substituted for elements thereof without departing from the scope of the invention. For example, while the finding phase and the tracking phase is described above with reference to health monitoring and tracking of the renal flow of patient 10, the finding phase and tracking phase can be used to monitor and track a blood flow of any organ in abdomen 40 of patient 10. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method for monitoring renal blood flow of a patient, the method comprising:
affixing an ultrasound transducer probe on an abdomen of the patient, wherein the ultrasound transducer probe comprises a two-dimensional array of transducer elements;
scanning, by the two-dimensional array of transducer elements and a beamformer driving the two-dimensional array of transducer elements, a volume of the abdomen of the patient under a field of view of the two-dimensional array of transducer elements;
identifying, by a processor in communication with the ultrasound transducer probe and the beamformer, a sub-volume in the volume with a Doppler flow signal comprising a signature of interest corresponding to the renal blood flow of the patient; and
tracking the sub-volume by periodically firing a sequential set of beams from the two-dimensional array of transducer elements over the sub-volume.

2. The method of claim 1, wherein scanning, by the two-dimensional array of transducer elements and the beamformer driving the two-dimensional array of transducer elements, the volume of the abdomen of the patient under the field of view of the two-dimensional array of transducer elements comprises:
scanning, by the two-dimensional array of transducer elements and the beamformer driving the two-dimensional array of transducer elements, the volume in a first dimension and in a second dimension only to determine a location of the renal blood flow of the patient in the first dimension and the second dimension; and
scanning, by the two-dimensional array of transducer elements and the beamformer driving the two-dimensional array of transducer elements, the volume in a third dimension only at the location of the renal blood flow of the patient in the first dimension and the second dimension to determine a location of the renal blood flow of the patient in the third dimension; wherein the third dimension is a depth dimension extending into the abdomen of the patient;
wherein the two-dimensional array of transducer elements scans the volume in the third dimension more frequently than the first dimension and the second dimension.

3. The method of claim 1, further comprising:
detecting, by the processor in communication with the ultrasound transducer probe and the beamformer, when a location of maximum intensity of the signature of interest is at an edge of the sequential set of beams and/or has a trajectory that is shifting the location of the maximum intensity of the signature of interest toward the edge of the sequential set of beams;
steering the sequential set of beams, by the beamformer driving the two-dimensional array of transducer elements, to maintain the location of the maximum intensity of the signature of interest at a center of the sequential set of beams; and
steering the sequential set of beams to a location based on a predicted future location of the maximum intensity of the signature of interest.

4. The method of claim 3, wherein steering the sequential set of beams to the location based on the predicted future location of the maximum intensity of the signature of interest comprises:
measuring estimates of a location of the renal blood flow by the beamformer;
inputting the estimates of the location of the renal blood flow into a predictive filter; and
determining, by the processor in communication with the ultrasound transducer probe and the beamformer, an expected trajectory of the location of the renal blood flow based on the estimates of the location of the renal blood flow and based on a breathing frequency of the patient.

5. The method of claim 4, wherein measuring estimates of the location of the renal blood flow by the beamformer comprises:
measuring, by the beamformer, differences in integrated power spectrum signal between individual beams of the set of sequential beams to estimate an azimuthal angle and an elevation angle of the location of the renal blood flow relative to the array of transducer elements; and
estimating, by the beamformer, a distance of the blood flow from the array of transducer elements in a distance dimension by:
gathering, by the array of transducer elements and the beamformer, a plurality of distance samples along a distance dimension;
calculating, by the beamformer, integrated power spectrum signal for each distance sample of the plurality of distance samples;
assigning, by the beamformer, a likelihood of containing the renal blood flow to each distance sample of the plurality of distance samples; and
calculating, by the beamformer, an estimate of a center of the renal blood flow from the plurality of distance samples.

6. The method of claim 5, wherein the method further comprises:
making, by the beamformer, proportional the likelihood of containing the renal blood flow to the integrated power spectrum signal for each distance sample of the plurality of distance samples; and
calculating, by the beamformer, the estimate of the center of the renal blood flow from the plurality of distance samples by selecting a distance sample of the plurality of distance samples with the largest integrated power spectrum signal.

7. The method of claim 4, further comprising:
measuring the breathing frequency of the patient with a breathing monitor connected to the patient; and
inputting the breathing frequency of the patient into the predictive filter from the breathing monitor.

8. The method of claim 4, further comprising:
measuring, by the beamformer, the breathing frequency of the patient by halting steering of the sequential set of beams for a period of time such that a position or positions of the sequential set of beams in the first dimension and the second dimension are fixed during the period of time;
observing, by the beamformer, a periodicity of integrated power spectrum signal of the sequential set of beams while the steering of the sequential set of beams is halted;
estimating the breathing frequency from the periodicity of the integrated power spectrum signal of the sequential set of beams while the steering of the sequential set of beams is halted; and
inputting, by the beamformer, the estimated breathing frequency of the patient into the predictive filter.

9. The method of claim 4, further comprising:
assigning over time, by the beamformer and/or the processor, an uncertainty estimate to new measurements of the estimates of the location of the renal blood flow based on a presence of a transient event, a signal-to-noise ratio of the integrated power spectrum signal, changes in moments of instantaneous spectrum of a Doppler flow signal of the renal blood flow, or changes in a maximum velocity envelope of a Doppler spectrogram of the renal blood flow; and
continuously inputting, from the beamformer and/or the processor to the predictive filter, the uncertainty estimate to adjust a degree or weight to which model parameters of the predictive filter are affected by new measurements during model parameter updates based upon the uncertainty estimate, such that the predictive filter can ignore measurements that are invalid.

10. The method of claim 4, further comprising:
determining, by the predictive filter, an estimate of the integrated power spectrum signal along each beam of the beam set based on a physical model of the location of the renal blood flow;
comparing, by the predictive filter, the estimate of the integrated power spectrum signal along each beam of the beam set to measurements of the integrated power spectrum signal along each signal beam of the beam set respectively; and
updating, by the processor in communication with the ultrasound transducer probe and the beamformer, model parameters of the physical model of the location of the renal blood flow based on differences between the estimate of the integrated power spectrum signal and the measurements of the integrated power spectrum signal along each signal beam of the beam set, wherein the model parameters of the physical model comprises beam shape of each beam of the beam set, vessel shape and orientation of a vessel containing the renal blood flow, and location of the vessel within the patient.

11. The method of claim 10, wherein determining, by the predictive filter, the estimate of the integrated power spectrum signal along each beam of the beam set based on the physical model of the location of the renal blood flow comprises:
calculating, by the physical model an overlap integral between the vessel and an ultrasound beam profile of each beam of the beam set; and
wherein the model parameters of the physical model comprise a description of how the ultrasound beam profile of each beam of the beam set changes with depth.

12. The method of claim 4, wherein the predictive filter comprises a Kalman Filter.

13. The method of claim 12, wherein:
the sequential set of beams comprises a central beam directed to the location of the maximum intensity of the signature of interest by the beamformer, and wherein the sequential set of beams comprises surrounding beams directed at the sub-volume by the beamformer that surround the central beam; an intensity of each beam of the sequential set of beams at a maximum intersection point between adjacent beams of the beam set is 3 dB to 6 dB; and
the central beam is fired more frequently than the surrounding beams to increase a signal-to-noise ratio (SNR) of the measured Doppler flow signal.

14. The method of claim 1, wherein the signature of interest of the Doppler flow signal comprises at least one of signal intensity of the Doppler flow signal, signal velocity of the Doppler flow signal, spectral shift of the Doppler flow signal, signal direction of the Doppler flow signal, spectral shift of signals surrounding the Doppler flow signal, and signal direction of the signals surrounding the Doppler flow signal.

15. The method of claim 1, further comprising:
limiting, by the beamformer, scanning of the two-dimensional array of transducer elements to the sub-volume while tracking the sub-volume by periodically firing the sequential set of beams from the two-dimensional array of transducer elements over the sub-volume.

16. The method of claim 1, further comprising:
defining, by the processor, dimensions of the sub-volume to contain the signature of interest of the Doppler flow signal, wherein the signature of interest has an intensity decay of 3 dB to 12 dB at edges of the sub-volume.

17. The method of claim 1, wherein scanning, by the two-dimensional array of transducer elements and the beamformer driving the two-dimensional array of transducer elements, the volume of the abdomen of the patient under the field of view of the two-dimensional array of transducer elements comprises:
dividing, by the processor, an entirety of the field of view of the two-dimensional array of transducer elements into multiple subsections, wherein each subsection of the multiple subsections overlaps in area by 25% to 33% with adjacent subsections;
focusing, by the two-dimensional array of transducer elements and the beamformer driving the two-dimensional array of transducer elements, multiple beams on each subsection of the multiple subsections for a period to form a Power Doppler Imaging (PDI) map of the volume; and
repeating in succession the focusing of the multiple beams on each subsection of the multiple subsections over an observation period to generate an accumulated PDI map of the volume.

18. The method of claim 1, wherein scanning, by the two-dimensional array of transducer elements and the beamformer driving the two-dimensional array of transducer elements, the volume of the abdomen of the patient under the field of view of the two-dimensional array of transducer elements comprises:
scanning, by the two-dimensional array of transducer elements and the beamformer driving the two-dimensional array of transducer elements, an entirety of the field of view of the two-dimensional array of transducer elements for multiple times over multiple respiration cycles of the patient to form a Power Doppler Imaging (PDI) map of the volume.

19. The method of claim 1, further comprising:
rescanning, by the two-dimensional array of transducer elements and the beamformer driving the two-dimensional array of transducer elements, the volume of the abdomen of the patient under the field of view of the two-dimensional array of transducer elements if the signature of interest of the Doppler flow signal falls below a preset threshold, criterium, criteria, and/or heuristic while tracking the sub-volume;
reidentifying, by the processor in communication with the ultrasound transducer probe and the beamformer, the sub-volume in the volume with the Doppler flow signal comprising the signature of interest corresponding to the renal blood flow of the patient; and
sending a signal, by the processor, to a display indicating that the signature of interest cannot be found and that the ultrasound transducer probe needs to be repositioned on the abdomen of the patient when the processor cannot identify in the volume the Doppler flow signal comprising the signature of interest above the preset threshold, criterium, criteria, and/or heuristic.

20. The method of claim 1, further comprising:
estimating, by the processor, a location of the Doppler flow signal comprising the signature of interest in the volume by calculating a centroid or mean of the Doppler flow signal comprising the signature of interest from each beam in the sequential set of beams.

21. The method of claim 1, further comprising:
utilizing, by the processor, a weighted average sum of beams in the sequential set of beams to maximize a signal-to-noise ratio (SNR) of the Doppler flow signal.

22. The method of claim 1, wherein beams in the sequential set of beams overlap with one another.

23. The method of claim 1, wherein the sequential set of beams is precalculated and stored in system memory in communication with the beam former and the processor, wherein a precalculated table in the system memory links the sequential set of beams with a coverage area of the sequential set of beams, and wherein the precalculated table links the sequential set of beams with a neighboring beam and/or a neighboring beam set and a coverage area of the neighboring beam and/or the neighboring beam set.

24. The method of claim 1, further comprising:
synthetically forming additional beams for the sequential set of beams to increase a granularity and/or signal-to-noise ratio (SNR) of the Doppler flow signal while tracking the sub-volume.
